(19) United States
(12) Patent Application Publication
(10) Pub. No.: US 2023/0248003 A1
(43) Pub. Date: Aug. 10, 2023

(54) BIOINSECTICIDE

(71) Applicant: Kannar Earth Science, Ltd., Lawrenceville, GA (US)

(72) Inventors: Samuel L. Cloete, Suwanee, GA (US); Justin Kwiatkowski, Atlanta, GA (US)

(21) Appl. No.: 18/163,977

(22) Filed: Feb. 3, 2023

Related U.S. Application Data
(63) Continuation of application No. PCT/US21/44921, filed on Aug. 6, 2021.
(60) Provisional application No. 63/215,239, filed on Jun. 25, 2021.
(60) Provisional application No. 63/210,086, filed on Jun. 14, 2021.
(60) Provisional application No. 63/061,906, filed on Aug. 6, 2020.

(51) Int. Cl. A01N 63/22; A01P 7/04

(52) U.S. Cl. CPC A01N 63/22; A01P 7/04

(57) ABSTRACT

A strain of Bacillus licheniformis (designated ‘K-357’) functions as an insecticide. Surprisingly, application of this single strain of B. licheniformis K-357 reduced nematode hatching and increased nematode mortality in vitro and reduced nematode counts and increased plant vigor and yield in cotton field trials. Nematicidal activity and cotton yield were compared for seed treated with B. licheniformis K-357 to in-furrow application of Aldicarb and Fluopyram, and seed treatment with Abamectin as nematicide. The nematicidal activity of the B. licheniformis K-357 was on par with, and in some cases superior to, that of the chemical treatments. The increases in cotton yield for seed treated with K-357 as nematicide were superior to that of both in-furrow application and seed treatment with the chemical nematicides. Corn seed treated with B. licheniformis K-357 in combination with chemical insecticides and fungicides had decreased root node injury in corn field trials heavily infested with corn rootworm.

### CROSS-REFERENCE TO RELATED APPLICATION

This application is a continuation of PCT patent application no. PCT/US2021/044921, filed on Aug. 6, 2021, which claims the benefit of priority of U.S. provisional patent application No. 63/061,906, filed on Aug. 6, 2020, U.S. provisional patent application No. 63/210,086, filed on Jun. 14, 2021, and U.S. provisional patent application No. 63/215,239, filed on Jun. 25, 2021, the contents of all of which are incorporated herein in their entirety by this reference.

### TECHNICAL FIELD

The present invention relates to use of a microbial composition for controlling plant pathogenic insects and enhancing crop yield. More specifically, the present invention relates to applying to the plant, a seed from which the plant is grown, or to an environment in which the plant is grown, a composition comprising a microbial strain.

### BACKGROUND

Crop responses to climate warming suggest that yields will decrease as growing-season temperatures increase. Deutsch et al. show that this effect may be exacerbated by insect pests (C. Deutsch, et al. Dryad (2018), https://dx.doi.org/10.5061/dryad.b7q3g2q). Insects already consume 5 to 20% of major grain crops. The authors' models show that for the three most important grain crops—wheat, rice, and maize—yield lost to insects will increase by 10 to 25% per degree Celsius of warming, hitting hardest in the temperate zone. These findings provide an estimate of further potential climate impacts on global food supply and a benchmark for future regional and field-specific studies of crop-pest-climate interactions.

Across the U.S. Corn Belt, corn rootworm (Diabrotica spp.) represents the number one group of insects that can threaten farmers' corn yields. Research estimates charted over the years place annual economic losses from these pests at close to $1 billion annually. Corn rootworms comprise a three-species insect complex: Western corn rootworm, Northern corn rootworm and Southern corn rootworm. All species possess lifecycle similarities, with the four stages of egg, larvae, pupa and adult beetle. They each lay eggs that hatch as larvae (worms) to feed on the roots of corn plants.

In addition, potato virus Y (PVY) is one of the ten most important plant viruses of solanaceous crops because of its worldwide distribution and economic impacts. The most effective way of controlling PVY is to avoid the introduction of the virus into fields. But once a plant is infected, it remains infected for its life, serving as an inoculum source for the spread of PVY by aphids. Approximately 65 aphid species have been documented to vector or transmit PVY with varying degrees of efficiency (Pelletier, Y. et al. J. Econ. Entomol. 105, 1909-1914, doi:10.1603/ec12085 (2012)). The use of insecticides and horticultural mineral oils to control for aphids may reduce PVY spread within the field. Still, most aphid vectors of PVY do not colonize potatoes and therefore are not affected by systemic insecticides. Commercially available virus-resistant cultivars only confer moderate resistance to PVY. Hence, there is a need to develop new strategies to control the in-season spread of PVY.

According to an estimate, plant parasitic nematodes are causing much more damage annually compared to insect pests (Singh et al., Procedia Environmental Sciences 29 (2015) 215-216). The crop yield loss due to these tiny unseen pests in various countries is enormous. They caused a projected yield loss of 12.3% ($157 billion dollars) worldwide. Growers identified insect pests and other constraints as production problems but overlooked plant parasitic nematodes. Nematode diseases are difficult to control because of their hidden nature and, hence, are more often overlooked. Plant parasitic nematodes not only cause damage individually but also form disease-complexes with other micro-organisms and increase crop losses.

The chemical standard of effectiveness for protecting crops from nematodes is Aldicarb, a carbamate insecticide which is the active substance in the pesticide formerly sold under the trade name TEMIK. Its use is banned in Europe and it was banned by the Environmental Protection Agency in 2010 because of its high toxicity. A new Aldicarb pesticide named AGLOGIC 15G, was approved by the EPA in December 2011 and began entering the market in 2015. Aldicarb has been among the most widely used pesticides internationally and is also one of the most environmentally toxic. Aldicarb poisoning from agricultural water runoff has led to the destruction of healthy ecosystems and the irreversible poisoning of fertile agricultural land.

Largely because of the desire to replace synthetic fertilizers and chemical pesticides to improve crop health and limit negative environmental impacts, interest in biological products for control of pests in agriculture has increased recently. Research has demonstrated the economic benefits of biological products and credible performance in integrated crop management systems. Microbial products are one category of biological products derived from naturally occurring microorganisms such as bacteria and fungi. These products are live, microbial strains that can be seed-applied, used in-furrow, or sprayed on crops to grow along with plants to protect from pests and diseases, or to enhance plant productivity and fertility. Microbial products offer the potential to deliver sustainable, cost-effective solutions that can help increase yield while using less input. However, there remains much to develop in control of plant pests with microbials, and especially in control of nematode pests.

The list of bacterial and fungal bionematicides that have been used in integrated nematode management strategies include Aspergillus niger, Paecilomyces lilacinus, Trichodeerma harzianum, T. viride, Pochonia chlamydosporia, Pasteuria penetrans, Pseudomonas flurorescens, Bacillus firmus, B. thuringiensis, B. velzensis, B. mojavensis, and B. subtilis (Xiang et al., Plant Disease (2017) 101:774; Abd-Elgawad and Askary, Egyptian Journal of Biological Pest Control (2018) 28:74). In one example, European Patent No. EP2603086B1 describes use of Bacillus subtilis strain DSM17231 in conjunction with Bacillus licheniformis strain DSM17236, which has been sold under the trade name NEMIX, for control of phytonematodes. This combination of strains was shown to have satisfactory effect on the inhibition of motion of the nematode, while the effect on hatching was not significant, and only the chemical product TEMIK inhibited hatching. Another example is the product, MELOCON WG (CERTIS USA), for controlling soil nematodes. MELOCON WG is based on the fungus Paecilomyces lilacinus a parasite of all stages of development of common plant-infecting nematodes, especially the eggs and infectious juveniles.

As described above, numerous microbial strains have been tested as nematicides and several bionematicide products are available commercially. However, the existing bionematicides and bioinsecticides are insufficient to address the serious, worldwide threat to crops from plant parasitic nematodes and pathogenic insects.

Thus, there is an unmet need for improved microbial insecticides that can reduce crop damage from insects, including nematodes, corn rootworm, and aphids which are some of the most problematic insects without the toxic effects of chemical insecticides. The present disclosure provides such an improved microbial and methods of its application to reduce insect damage and improve plant health and crop yields.

### SUMMARY

In one embodiment, a method is provided for controlling insect damage to a plant, the method comprising: planting a plant or a seed of a plant in a suitable growth medium, the plant or the seed having a coating or partial coating of a composition comprising: a biologically pure culture of a Bacillus licheniformis K-357, or a mutant thereof having all the identifying characteristics thereof; and optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a method is provided for controlling insect damage to a plant, the method comprising: delivering to seed of a plant, foliage of a plant, roots of a plant, or soil or growth medium surrounding a plant, a composition comprising: a biologically pure culture of a Bacillus licheniformis K-357, or a mutant thereof having all the identifying characteristics thereof; and optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a method is provided for controlling insect damage to a plant, the method comprising: planting a plant or a seed of a plant in a suitable growth medium, the plant or the seed having a coating or partial coating of a composition consisting essentially of: a biologically pure culture of a Bacillus licheniformis K-357, or a mutant thereof having all the identifying characteristics thereof; and one or more carriers, excipients, or nutrients, wherein the composition improves plant yield or reduces plant insect damage in the presence of a plant pathogenic insect, or both.

In one embodiment, a method is provided for controlling insect damage to a plant, the method comprising: delivering to seed of a plant, foliage of a plant, roots of a plant, or soil or growth medium surrounding a plant, a composition consisting essentially of: a biologically pure culture of a Bacillus licheniformis K-357, or a mutant thereof having all the identifying characteristics thereof, and one or more carriers, excipients, or nutrients, wherein the composition improves plant yield or reduces plant insect damage in the presence of a plant pathogenic insect, or both.

In one embodiment, a method is provided for plant or plant seed treatment comprising applying to a plant or seeds of a plant a coating of a composition comprising a biologically pure culture of a Bacillus licheniformis K-357, or a mutant thereof having all the identifying characteristics thereof, the composition optionally comprising one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a plant or a seed of a plant is provided having a coating of a composition comprising: a biologically pure culture of a Bacillus licheniformis K-357 or a mutant thereof having all the identifying characteristics thereof; and optionally, one or more carriers, excipients, nutrients, or microbial-, biological- or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method is provided for plant or plant seed treatment comprising applying to a plant or seeds of a plant a coating of a composition comprising a biologically pure culture of a Bacillus licheniformis K-357, or a mutant thereof having all the identifying characteristics thereof, the composition optionally comprising one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a plant or a seed of a plant is provided having a coating of a composition comprising: a biologically pure culture of a Bacillus licheniformis K-357 or a mutant thereof having all the identifying characteristics thereof; and optionally, one or more carriers, excipients, nutrients, or microbial-, biological- or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment a method is provided for controlling plant pathogenic nematodes that includes planting a seed of a plant in a suitable growth medium, the seed having a coating including spores of a biologically pure culture of a single microbial strain, said single microbial strain is Bacillus licheniformis K-357, or a mutant thereof having all the identifying characteristics thereof and, optionally, one or more carriers, excipients, nutrients, crop protection agents, fungicides, insecticides, or nematicides. The Bacillus licheniformis K-357 is present on the seed in an amount suitable to improve plant yield in the presence of a plant pathogenic nematode, improve plant resistance to a plant pathogenic nematode, or reduce plant infection by a plant pathogenic nematode, and combinations thereof.

In one embodiment a method is provided for controlling plant pathogenic nematodes that includes delivering to seed of a plant, roots of a plant, or growth medium surrounding a plant, a composition including a biologically pure culture of a single microbial strain, said microbial strain is Bacillus licheniformis K-357, or a mutant thereof having all the identifying characteristics thereof and optionally, one or more carriers, excipients, nutrients, crop protection agents, fungicides, insecticides, or nematicides. Delivering of the composition improves plant yield in the presence of a plant pathogenic nematode, improves plant resistance to a plant pathogenic nematode, or reduces plant infection by a plant pathogenic nematode, and combinations thereof.

In one embodiment a method is provided for controlling plant pathogenic nematodes that includes delivering to seed of a plant, roots of a plant, or soil or growth medium surrounding a plant, a composition consisting essentially of a biologically pure culture of a Bacillus licheniformis K-357, or a mutant thereof having all the identifying characteristics thereof, and one or more carriers, excipients, or nutrients. Delivering of the composition improves plant yield in the presence of a plant pathogenic nematode, improves plant resistance to a plant pathogenic nematode, or reduces plant infection by a plant pathogenic nematode, and combinations thereof.

In one embodiment a method is provided of seed treatment that includes applying to seeds a coating of a biologically pure culture of a single microbial strain, said microbial strain is Bacillus licheniformis K-357, or a mutant thereof having all the identifying characteristics thereof, the coating optionally including one or more carriers, excipients, nutrients, crop protection agents, fungicides, insecticides, or nematicides. The Bacillus licheniformis K-357 is present on the treated seed in an amount suitable to improve plant yield in the presence of a plant pathogenic nematode, improve plant resistance to a plant pathogenic nematode, or reduce plant infection by a plant pathogenic nematode, and combinations thereof.

In one embodiment a plant seed is provided having a coating that includes a biologically pure culture of a single microbial strain, said single microbial strain is Bacillus licheniformis K-357 or a mutant thereof having all the identifying characteristics thereof and, optionally, one or more carriers, excipients, nutrients, crop protection agents, fungicides, insecticides, or nematicides. The Bacillus licheniformis K-357 is present on the seed in an amount suitable to improve plant yield in the presence of a plant pathogenic nematode, improve plant resistance to a plant pathogenic nematode, or reduce plant infection by a plant pathogenic nematode, and combinations thereof.

In one embodiment, a composition is provided for improving plant health and/or yield comprising: i) a biologically pure culture of a Bacillus licheniformis K-357, or a mutant thereof having all the identifying characteristics thereof; ii) soy protein hydrolysate; iii) chelated ferrous sulfate; and iv) optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a plant or a seed of a plant is provided having a coating of a composition comprising: i) a biologically pure culture of a Bacillus licheniformis K-357, or a mutant thereof having all the identifying characteristics thereof; ii) a hydrolysate of soy protein; iii) chelated ferrous sulfate; and iv) optionally one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method of plant or plant seed treatment is provided, the method comprising: applying to a plant or seeds of a plant a coating of a composition comprising: i) a biologically pure culture of a Bacillus licheniformis K-357, or a mutant thereof having all the identifying characteristics thereof; ii) a hydrolysate of soy protein; iii) chelated ferrous sulfate; and iv) optionally one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method is provided of formulating a microbial crop protection agent to improve plant health and/or yield, comprising: adding together a hydrolysate of soy protein, a chelated ferrous sulfate, and a biological culture of one or more microbial crop protection agents, wherein the one or more microbial crop protection agents are present in the formulation in an amount suitable to improve plant yield or reduce plant insect damage in the presence of a plant pathogenic insect, or both.

In one embodiment, a formulation of a microbial crop protection agent is provided to improve plant health and/or yield, comprising: i) a biological culture of one or more microbial crop protection agents; ii) a hydrolysate of soy protein; iii) chelated ferrous sulfate; and iv) optionally, one or more carriers, excipients, nutrients, or non-microbial crop protection agents, wherein the one or more microbial crop protection agents are present in an amount suitable to improve plant yield or reduce plant insect damage in the presence of a plant pathogenic insect, or both.

### BRIEF DESCRIPTION OF THE DRAWINGS

FIG. 1 is a graph showing the percentage of nematode hatch counts 10 days after in vitro application of varying dosage of B. licheniformis K-357 spores (0.0256 1 L/ha—corresponding to 5.5×10⁶ CFU in 50 ml; 0.1428 5 L/ha—corresponding to 2.75×107 CFU in 50 ml; 0.3333 10 L/ha—corresponding to 5.5×107 CFU in 50 ml) on species Heterodera glycines, Meloidogyne incognita, and Rotylenchulus reniformis.

FIG. 2A is an image of nematodes without addition of B. licheniformis K-357 showing nematode eggs, juveniles, and adults.

FIG. 2B is an image of nematodes without addition of B. licheniformis K-357 showing unhatched juveniles in eggs.

FIG. 2C is an image of nematodes without addition of B. licheniformis K-357 showing a hatching juvenile.

FIG. 2D is an image of nematodes without addition of B. licheniformis K-357 showing an adult nematode.

FIG. 2E is an image of nematodes without addition of B. licheniformis K-357 showing an adult nematode.

FIG. 2F is an image of nematodes without addition of B. licheniformis K-357 showing a mouth of an adult nematode.

FIG. 3 is an image showing B. licheniformis K-357 colonization of an adult nematode at 1000× magnification.

FIG. 4 is an image at 1000× magnification of B. licheniformis K-357 stained red, surrounding an immobile Reniform nematode egg.

FIG. 5 is an image at 1000× magnification of a Reniform egg surrounded by purple stained B. licheniformis K-357. The juvenile vermiform is visible inside the center of the egg and is immobile under microscopic evaluation.

FIG. 6 is a graph showing the percentage of reniform nematode counts in a field trial in Arkansas 55 days after emergence of cotton seed treated in-furrow with either chemical nematicide Aldicarb or Fluopyram as compared to seed treated with K-357: 1) seed treated with fungicides (Azoxystrobin, Fludioxinol, Mefenoxam, Sedaxane), insecticides (Thiamethoxam), and in-furrow nematicide Aldicarb (15 lbs/acre), 2) seed treated with fungicides (Azoxystrobin, Fludioxinol, Mefenoxam, Sedaxane), insecticides (Thiamethoxam, Imidacloprid), and in-furrow nematicide Fluopyram (16 oz/acre), and 3) seed treated with fungicides (Metalaxyl, Fludioxinol, Myclobutanil), insecticide (Imidacloprid), and K-357 as nematicide at two fluid ounces per hundred pounds of cotton seed (“2.000 Fl. Oz./CWT”).

FIG. 7 is a graph showing the percentage of cotton yield at harvest for the crop of FIG. 6.

FIG. 8 is a graph showing the root knot nematode juvenile population at planting, after 60 days, and at harvest in a cotton field trial conducted in Texas comparing seed treated with Abamectin as compared to seed treated with K-357 as nematicide, specifically, seed treated with: 1) fungicides (Azoxystrobin, Fludioxinol, Mefenoxam, Sedaxane), insecticide (Imidacloprid, Thiamethoxam), and AVICTA ELITE PLUS with VIBRANCE (Abamectin) as nematicide; or 2) fungicides (Metalaxyl, Fludioxinol, Myclobutanil), insecticide (Imidacloprid), and two fluid ounces per hundred pounds of cotton seed (2.000 Fl. Oz./CWT) of K-357 as nematicide.

FIG. 9 is a graph showing the percentage of nematode count in a field trial in Mississippi 25 days after planting cotton seed treated with fungicides (Azoxystrobin, Fludioxinol, Mefenoxam, Sedaxane), insecticides (Imidacloprid, Thiamethoxam), and Abamectin as nematicide, as compared to seed treated with the fungicides (Metalaxyl, Fludioxinol, Myclobutanil), insecticide (Imidacloprid), and with either two (“2.000 Fl. Oz./CWT) or three (3.000 Fl. Oz./CWT) fluid ounces per hundred pounds of cotton seed of K-357 as nematicide.

FIG. 10 is a graph showing cotton yield at harvest for the crop of FIG. 9.

FIG. 11 is a graph showing plant vigor calculated on a scale of 1-9, 12 and 25 days after planting cotton seed in the field trial of FIG. 9.

FIG. 12 is graph showing the percentage of nematode mortality for each of 4 species of plant pathogenic nematodes treated in vitro with either Avicta or K-357 with water as a control.

FIG. 13 is a graph showing corn mean stand count 37 days after planting at the University of Illinois, Monmouth site, in fields heavily infested with CRW for corn seed treated with K-357 in addition to chemical insecticides/fungicides at the label rate as compared to corn seed treated with chemical insecticides/fungicides alone.

FIG. 14 is a graph showing corn root node-injury evaluation mean score 64 days after planting at the University of Illinois, Monmouth site, in fields heavily infested with CRW for corn seed treated with K-357 in addition to chemical insecticides/fungicides at the label rate as compared to corn seed treated with chemical insecticides/fungicides alone.

FIG. 15 is a schematic illustrating the successive steps of an experimental set-up to investigate the impact of B. licheniformis K-357 on the transmission of potato virus Y (PVY) by the green peach aphid (GPA).

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used herein, the phrase “a biologically pure culture” includes one or a combination of spores and vegetative cells of the biologically pure fermentation culture of the microbial strain. By “biologically pure” is meant essentially biologically pure as it is understood in the art. In addition, “biologically pure culture” includes a mutant of the microbial strain having all the identifying characteristics thereof.

Following long-standing patent law convention, the terms “a,” “an,” and “the” refer to “one or more” when used in this application, including the claims. Thus, for example, reference to “a plant or a seed of a plant” includes a plurality of plants or seeds, unless the context clearly is to the contrary, and so forth.

Throughout this specification and the claims, the terms “comprise,” “comprises,” and “comprising” are used in a non-exclusive sense, except where the context requires otherwise. Likewise, the terms “having” and “including” and their grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range. In addition, as used herein, the term “about”, when referring to a value can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed compositions and methods. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Throughout this specification and the claims, the phrase “microbial-, biological-, or chemical-crop protection agents” means any microbial, biological, or chemical agent that functions to protect crops against plant pests and/or plant pathogens including agents that function as one or more of a biocide, an insecticide, a fungicide, or a nematicide.

For the purposes of this specification and claims, the term “insect” includes nematodes.

For the purposes of the specification and claims, the phrase “foliage of a plant” means any plant part other than seed and roots.

For the purposes of the specification and claims, the term “pesticide” and “insecticide” are used interchangeably.

Due to a desire to replace or reduce synthetic fertilizers and chemical pesticides to improve crop health and limit negative environmental impacts, a live, microbial strain, and compositions comprising, and seeds and plants coated therewith, are herein provided to control plant parasitic nematodes and other pathogenic insects including, but not limited to, aphids and corn rootworm. Research has demonstrated the economic benefits of such live, microbial products and credible performance in integrated crop management systems. The live, microbial strain provided herein can be seed-applied, used in-furrow, or applied on plant tips or roots to grow along with plants to protect from nematode and insect pests and/or to enhance plant productivity and fertility. The live, microbial products and associated methods provided herein offer the potential to deliver sustainable, cost-effective solutions that can increase yield with less input, while avoiding the toxic effects on users and the environment associated with chemical alternatives.

Bacillus licheniformis strain NRRL B-23318, originally isolated from the Sonoran Desert, was obtained from the NRRL culture collection. Surprisingly, the present inventors discovered that this strain of Bacillus licheniformis (designated herein as ‘K-357’) can function as a bionematicide. More specifically, application of B. licheniformis K-357 reduced nematode hatching in vitro and reduced nematode soil counts and increased cotton yield in field trials. The nematicidal activity of the B. licheniformis K-357 was on par with or significantly superior to that of standard chemical nematicide treatments. The increases in cotton yield with B. licheniformis K-357 were superior to that of standard chemical treatments. Exemplary experiments and data are described herein.

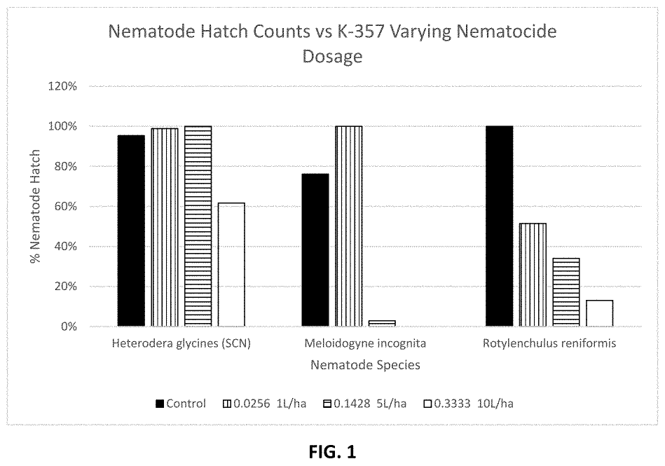

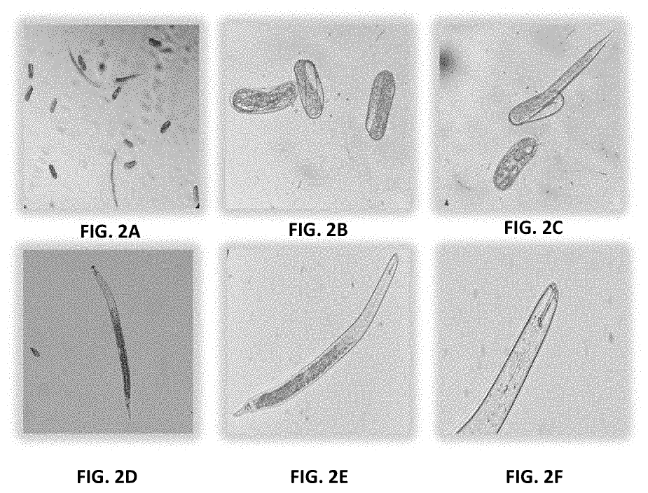

Example 1 describes an in vitro experiment in which the effect is examined of B. licheniformis K-357 on control of three different types of phytonematodes: Heterodera glycines, Meloidogyne incognita, and Rotylenchulus reniformis. The results shown in FIG. 1 illustrate that K-357 can reduce or prevent nematode hatching in a dose-dependent manner, including an 87% reduction of R. reniformis and complete inhibition of M. incognita hatching. FIGS. 2-5 show K-357 colonizing the nematodes and nematode eggs. The substantial K-357 inhibition/prevention of hatching on R. reniformis/M. incognita is superior to that observed for in vitro application of Bacillus subtilis strain DSM17231 in conjunction with Bacillus licheniformis strain DSM17236, where a lessor effect was observed for only one of three Meloidogyne species tested (see EP2603086B1). What makes the K-357 findings described herein even more surprising, is that it is generally accepted by those of skill in this field that any bionematicidal effects exerted by the combination of strains described in EP2603086B1 (i.e., B. subtilis strain DSM17231+B. licheniformis strain DSM17236) result from the B. subtilis strain rather than from the B. licheniformis strain, as there are no known B. licheniformis strains reported to have nematicidal properties (see, e.g., Xiang et al. (2017); Abd-Elgawad and Askary (2018)).

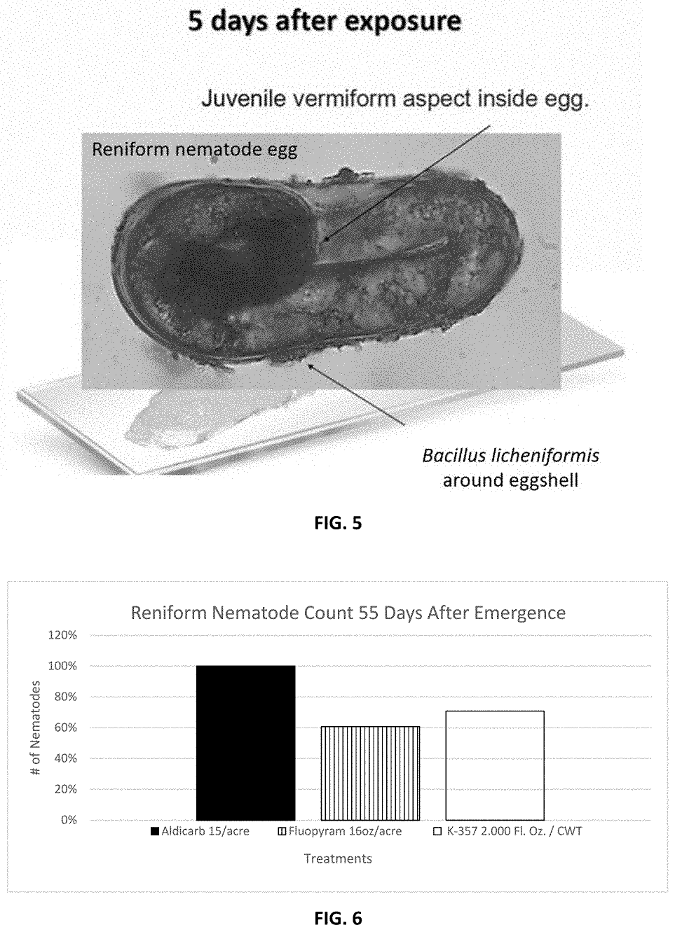

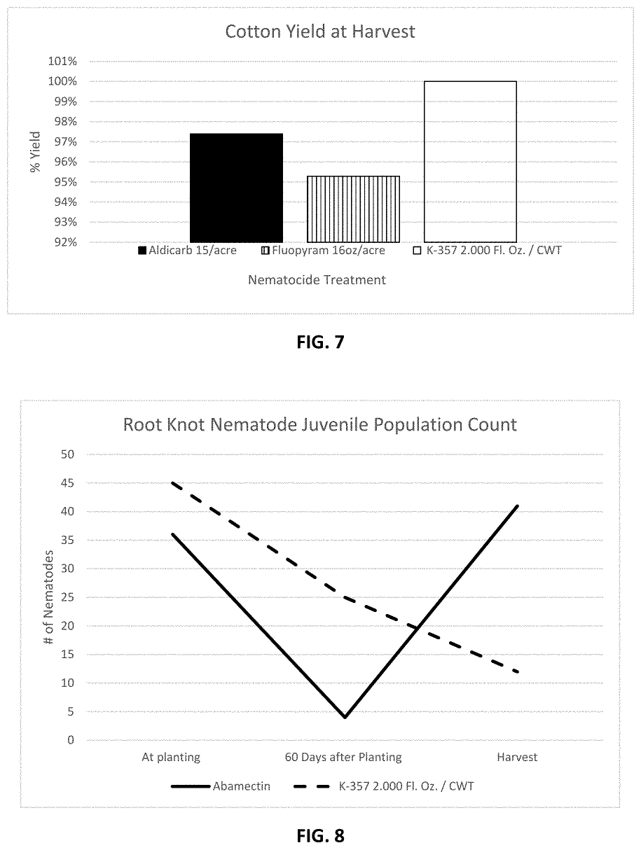

Example 2 describes field trials of cotton to evaluate the efficacy of B. licheniformis K-357 compared to chemical nematicides including Aldicarb, which is the standard of effectiveness. Each of the treatments were applied in-furrow at the time of seed planting. The results are shown in FIGS. 6 and 7. The results show that K-357 reduced nematode count in the soil 55 days after emergence to about 70% of the chemical standard, Aldicarb, and out-performed Fluopyram. FIG. 7 is a graph showing the percentage of cotton yield at harvest. Surprisingly, B. licheniformis K-357 resulted in a higher yield of cotton than both Fluopyram (˜4.5% higher) and Aldicarb (˜2.5% higher).

Example 3 describes field trials of cotton to evaluate the efficacy of B. licheniformis K-357 when applied to seed in reducing nematode count and increasing yield. In the trial, seed treated with two different rates of B. licheniformis K-357 was compared to seed treated with Abamectin. The results are shown in FIGS. 8 through 11. FIG. 8 shows that K-357 has a more enduring effect on inhibition of root knot nematode juvenile populations than the chemical nematicide Abamectin. Specifically, while the nematode population was lower for Abamectin treated than for K-357 after 60 days, this relationship was reversed by the time of harvest. At harvest the nematode count was reduced to less than 15 for K-357 as opposed to about 40 for Abamectin. In addition, the nematode population started out higher in the K-357 group than in the Abamectin group and steadily decreased over the course of the trial. In contrast, the nematode count for the Abamectin group reversed and grew rapidly higher after 60 days.

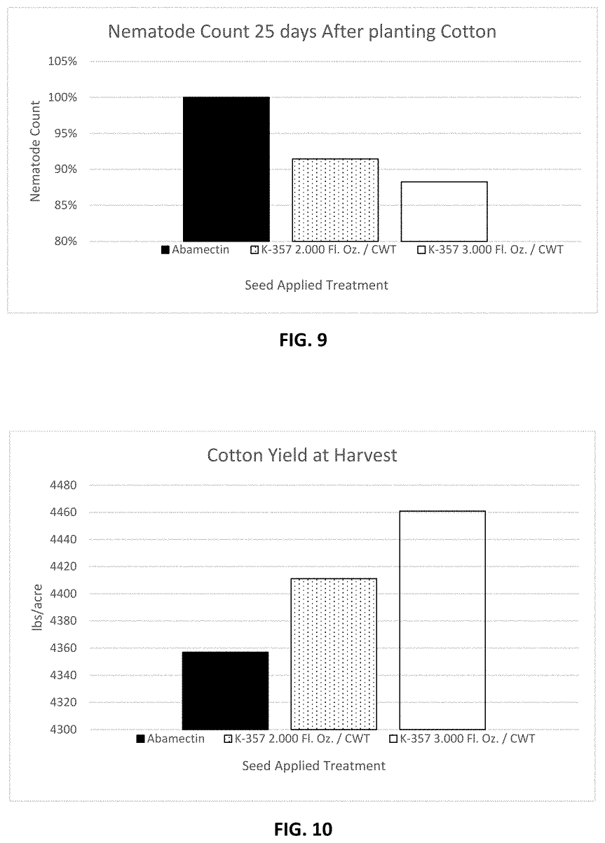

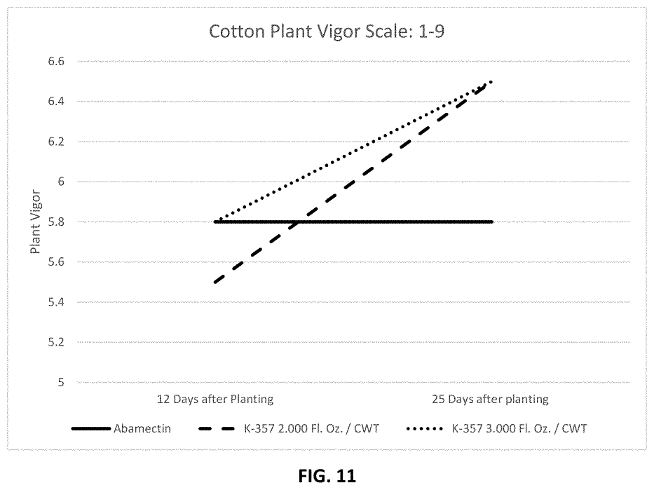

FIG. 9 illustrates that the nematode count 25 days after planting the treated cotton seed was controlled 88% and 91% as effectively by seed treatment with 2-oz/cwt and 3-oz/cwt of K-357, respectfully, than for seed treated with Abamectin. FIG. 10 illustrates that treatment with 2- and 3.000 Fl. Oz./CWT B. licheniformis K-357 resulted in a surprisingly significant increase in cotton yield of 54 and 102 lbs/acre, respectively. In addition, FIG. 11 shows that K-357 improved plant vigor between 12 and 25 days after planting, whereas no improvement in vigor was observed for Abamectin.

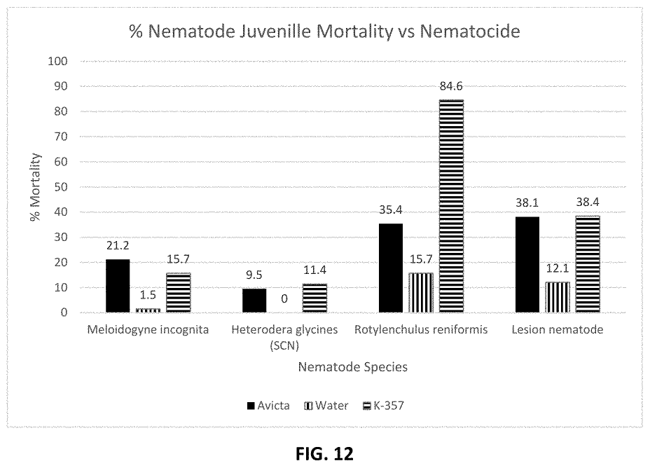

Example 4 describes an in vitro experiment examining the effect of B. licheniformis K-357 on four different types of phytonematodes: Heterodera glycines, Meloidogyne incognita, Rotylenchulus reniformis, and Lesion nematode as compared to the chemical nematicide Avicta. Nematodes were placed in petri dishes and treated with Avicta, water, or K-357 spores for 48 hours. The data are shown in FIG. 12, which is a graph showing the percentage of nematode mortality for each of the 4 species. Surprisingly, the K-357 treatment was equally or substantially more effective at killing nematodes than Avicta. Specifically, K-357 was equally effective at killing Meloidogyne incognita, Heterodera glycines, and Lesion nematode, and substantially better at killing Rotylenchulus reniformis than Avicta (84.6% versus 35.4%, respectively).

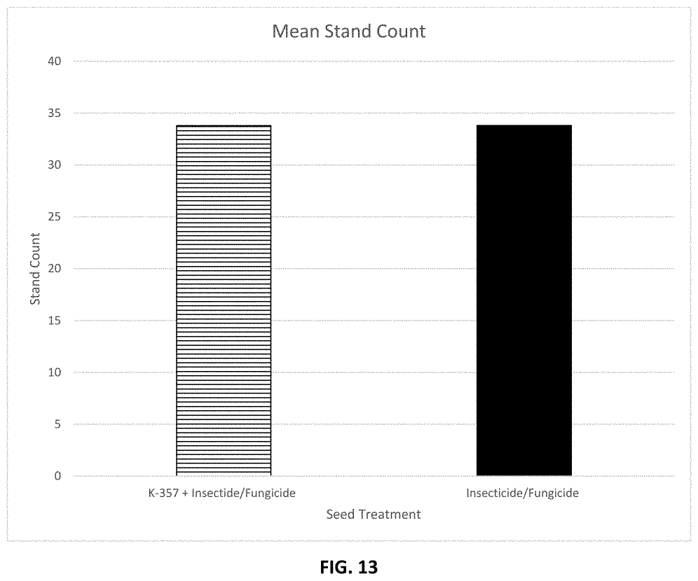

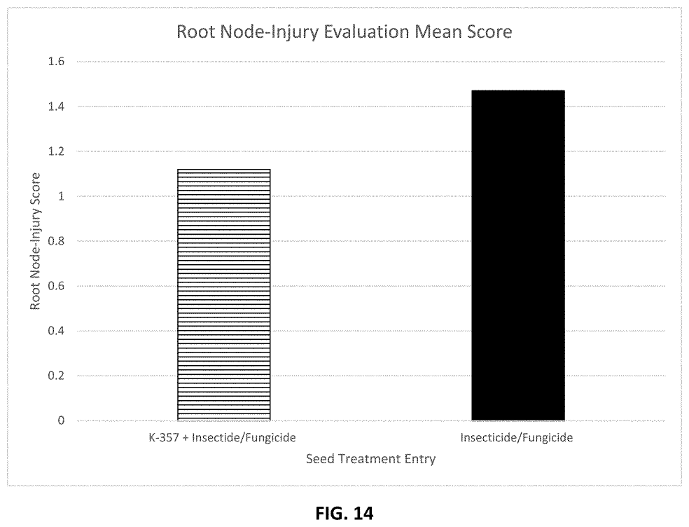

In addition to nematodes, the B. licheniformus K-357 can have pesticidal activity against other plant pathogenic insects. Example 5 describes a field trial examining of seed treated with B. licheniformis K-357 alone and in combination with chemical insecticides and fungicides for the ability to reduce corn rootworm (CRW) damage and increase yield. The trials were performed at the University of Illinois, Monmouth site, in fields heavily infested with CRW. The results of stand counts and root node injury analysis 37 days and 64 days after planting are shown in FIGS. 13 and 14, respectively. The mean stand count for seed treatment group #1 (Insecticide/Fungicide) and seed treatment group #2 (K-357+Insecticide/Fungicide) were both 33.8 (FIG. 13). However, the root node injury graph in FIG. 14 shows that the addition of K-357 to the Insecticide/Fungicide mixture decreased root node injury. Specifically, the root node injury score for seed treatment group #2 (K-357+Insecticide/Fungicide) was 1.12 compared to 1.47 for seed treatment group #1 (Insecticide/Fungicide).

Another example of the B. licheniformus K-357 having insecticidal activity is described in Example 6. In this experiment, B. licheniformis K-357 is evaluated for its ability to reduce or prevent potato virus Y (PVY) transmission using a virus transmission assay on Nicotiana benthamiana detached leaves. The green peach aphid (GPA, Myzus persicae) is the most efficient vector of PVY and the experiment is designed to access whether B. licheniformus K-357 can debilitate hemptipteran pests such as aphids resulting in complete inhibition and/or reduction of PVY transmission.

In one embodiment, a method is provided for controlling insect damage to a plant, the method comprising: planting a plant or a seed of a plant in a suitable growth medium, the plant or the seed having a coating or partial coating of a composition comprising: a biologically pure culture of a Bacillus licheniformis K-357, or a mutant thereof having all the identifying characteristics thereof; and optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a method is provided for controlling insect damage to a plant, the method comprising: delivering to seed of a plant, foliage of a plant, roots of a plant, or soil or growth medium surrounding a plant, a composition comprising: a biologically pure culture of a Bacillus licheniformis K-357, or a mutant thereof having all the identifying characteristics thereof; and optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a method is provided for controlling insect damage to a plant, the method comprising: planting a plant or a seed of a plant in a suitable growth medium, the plant or the seed having a coating or partial coating of a composition consisting essentially of: a biologically pure culture of a Bacillus licheniformis K-357, or a mutant thereof having all the identifying characteristics thereof; and one or more carriers, excipients, or nutrients, wherein the composition improves plant yield or reduces plant insect damage in the presence of a plant pathogenic insect, or both.

In one embodiment, a method is provided for controlling insect damage to a plant, the method comprising: delivering to seed of a plant, foliage of a plant, roots of a plant, or soil or growth medium surrounding a plant, a composition consisting essentially of: a biologically pure culture of a Bacillus licheniformis K-357, or a mutant thereof having all the identifying characteristics thereof, and one or more carriers, excipients, or nutrients, wherein the composition improves plant yield or reduces plant insect damage in the presence of a plant pathogenic insect, or both.

In one embodiment, a method is provided for plant or plant seed treatment comprising applying to a plant or seeds of a plant a coating of a composition comprising a biologically pure culture of a Bacillus licheniformis K-357, or a mutant thereof having all the identifying characteristics thereof, the composition optionally including one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a plant or a seed of a plant is provided having a coating of a composition comprising: a biologically pure culture of a Bacillus licheniformis K-357 or a mutant thereof having all the identifying characteristics thereof; and optionally, one or more carriers, excipients, nutrients, or microbial-, biological- or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method is provided for plant or plant seed treatment comprising applying to a plant or seeds of a plant a coating of a composition comprising a biologically pure culture of a Bacillus licheniformis K-357, or a mutant thereof having all the identifying characteristics thereof, the composition optionally including one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a plant or a seed of a plant is provided having a coating of a composition comprising: a biologically pure culture of a Bacillus licheniformis K-357 or a mutant thereof having all the identifying characteristics thereof; and optionally, one or more carriers, excipients, nutrients, or microbial-, biological- or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment of the present disclosure, a method is provided for controlling plant pathogenic nematodes. The method includes delivering to seed of a plant, roots of a plant, or soil or growth medium surrounding a plant, a composition consisting essentially of a biologically pure culture of a Bacillus licheniformis K-357, or a mutant thereof having all the identifying characteristics thereof, and one or more carriers, excipients, or nutrients. Delivering the composition to the plant seed, roots, or growth medium can improve one or both of plant yield in the presence of a plant pathogenic nematode and plant resistance to a plant pathogenic nematode. In some cases, delivering the composition can also reduce plant infection by a plant pathogenic nematode.

What is meant by “a composition consisting essentially of” a biologically pure culture of a Bacillus licheniformis K-357 is that the composition includes only a single biologically pure culture of microbial strain, which is B. licheniformis K-357. Thus, the phrase “consisting essentially of” is only meant to refer to microbial strains, i.e. that the microbial strains included in the composition are limited to the strain B. licheniformis K-357. While the composition only contains the single microbial strain B. licheniformis K-357, the composition may contain any number of other non-microbial strain ingredients including, but not limited to, one or more carriers, excipients, nutrients, crop protection agents, insecticides, nematicides, or fungicides.

In one embodiment of the present disclosure, a method is provided for controlling plant pathogenic nematodes, which includes delivering to seed of a plant, roots of a plant, or growth medium surrounding a plant, a composition including a biologically pure culture of a single microbial strain, said single microbial strain is Bacillus licheniformis K-357, or a mutant thereof having all the identifying characteristics thereof. Delivering the composition to the plant seed, roots, or growth medium can improve one or both of plant yield in the presence of a plant pathogenic nematode and plant resistance to a plant pathogenic nematode. In some cases, delivering the composition can also reduce plant infection by a plant pathogenic nematode.

Similar to that described above, the “composition including a biologically pure culture of a single microbial strain, said single microbial strain is Bacillus licheniformis K-357” is intended to mean that, while the composition can include an unlimited number of ingredients, the microbial strain ingredients are limited to the biologically pure culture of the single strain B. licheniformis K-357. The composition may contain any number of other non-microbial ingredients including, but not limited to, one or more carriers, excipients, nutrients, crop protection agents, insecticides, nematicides, or fungicides.

In one embodiment of the present disclosure, a method is provided for controlling plant pathogenic nematodes, which includes planting a coated seed in a suitable growth medium. In this example, the seed has a coating that includes spores of a biologically pure culture of a single microbial strain, said single microbial strain is Bacillus licheniformis K-357, or a mutant thereof having all the identifying characteristics thereof; and optionally, one or more carriers, excipients, nutrients, crop protection agents, fungicides, insecticides, or nematicides. The B. licheniformis K-357 is present in an amount suitable for one or more of improving plant yield in the presence of a plant pathogenic nematode, improving plant resistance to a plant pathogenic nematode, or reducing plant infection by a plant pathogenic nematode. The composition that the seed has been coated with can include one or more carriers, excipients, or nutrients as well as one or more crop protection agents, insecticides, nematicides, or fungicides.

The Bacillus licheniformis K-357 can be present on the seed in an amount ranging from 1.0×10¹² CFU/g to 1.0×10¹ CFU/g, from 9.0×10⁷ CFU/g to 2.0×10⁴ CFU/g, or from 4.0×10⁸ CFU/g to 1.0×10⁴ CFU/g seed.

The compositions of the present disclosure that include the B. licheniformis K-357 can be in the form of a liquid. The B. licheniformis K-357 in the liquid compositions can be present at a concentration ranging from about 1.0×10¹² to 1.0×10¹ CFU/ml, 1.0×10¹¹ CFU/ml to 1.0×10² CFU/ml, or from 1.0×10¹⁰ CFU/ml to 1.0×10⁴ CFU/ml.

The compositions of the present disclosure that include the B. licheniformis K-357 can also be in the form of a dust, a dry wettable powder, a spreadable granule, or a dry wettable granule. The B. licheniformis K-357 in these non-liquid forms can be present in an amount ranging from 1.0×10¹² CFU/g to 1.0×10¹⁰ CFU/g, from 5.0×10¹¹ CFU/g to 1.0×10³ CFU/g, or from 1.25×10¹¹ CFU/g to 1.0×10⁸ CFU/g.

In one embodiment of the present disclosure, a method is provided for seed treatment that includes applying to seeds a coating of a biologically pure culture of a single microbial strain, said single microbial strain is Bacillus licheniformis K-357, or a mutant thereof having all the identifying characteristics thereof. While the coating is limited to a biologically pure culture of the single microbial strain, B. licheniformis K-357, the coating optionally may include one or more carriers, excipients, nutrients, crop protection agents, fungicides, insecticides, or nematicides. The Bacillus licheniformis K-357 is present in the coating in an amount suitable to improve plant yield in the presence of a plant pathogenic nematode, improve plant resistance to a plant pathogenic nematode, and/or reduce plant infection by a plant pathogenic nematode.

The embodiments of the present disclosure also include plant seed having a coating comprising a biologically pure culture of a single microbial strain, said single microbial strain is Bacillus licheniformis K-357 or a mutant thereof having all the identifying characteristics thereof. Optionally, the seed coating may also include one or more crop protection agents, including, but not limited to, insecticides, nematicides, fungicides, or nutrients, or other carriers or excipients. The Bacillus licheniformis K-357 is present in the coating on the seed in an amount suitable to improve plant yield in the presence of a plant pathogenic nematode, improve plant resistance to a plant pathogenic nematode, and/or reduce plant infection by a plant pathogenic nematode.

In the methods and compositions of the present disclosure, the plant can be a monocot or a dicot, or any one of, but not limited to, cotton, rice, soybean, tomato, cereals, root/tuber and corn vegetables, brassica vegetables, cucurbit vegetables, bulb vegetables, citrus, fruiting vegetables, herbs/spices, leafy vegetables, legumes/vegetables (succulent and dried beans and peas), oil seed crops, pome fruit, stone fruit, strawberry, sugarcane, sugarbeet, tree nuts, kiwi, banana, grass, ornamental plants, or hardwood cuttings.

In one example, the plant or the plant seed having the coating comprising the Bacillus licheniformis K-357, or a mutant thereof having all the identifying characteristics thereof, is corn, cotton, peanuts, soy beans, sorghum, wheat, rice, potato, sunflower, or onion.

In one embodiment of methods and compositions of the present disclosure, plant root tips or transplant roots can be coated with the composition comprising the Bacillus licheniformis K-357, or a mutant thereof having all the identifying characteristics thereof.

In one embodiment of the present disclosure, the compositions can be applied to seed of a plant, foliage of a plant, roots of a plant, or growth medium surrounding a plant.

In the methods and compositions of the present disclosure, the plant pathogenic nematode can include, but is not limited to, the species Meioidogyne incognita, M. paranaensis. M. javonica, Heterodera glycines (SCN), Heterodera glycines (SCN), Rotylenchulus reniformis, or Lesion nematode.

In one embodiment of the methods and compositions of the present disclosure, the pathogenic insect can include, but is not limited to, corn rootworm, western corn rootworm (WCR), northern corn rootworm (NCR), southern corn rootworm (SCR), aphids, Green peach aphid (Myzus persicae), or Cotton aphid (Aphis gossypii).

In some embodiments, the Bacillus licheniformis K-357 is formulated with a soy protein hydrolysate and chelated ferrous sulfate.

For example, in one embodiment, a composition is provided for improving plant health and/or yield comprising: i) a biologically pure culture of a Bacillus licheniformis K-357, or a mutant thereof having all the identifying characteristics thereof; ii) soy protein hydrolysate; iii) chelated ferrous sulfate; and iv) optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a composition is provided for improving plant health and/or yield comprising: i) a biologically pure culture of a Bacillus licheniformis K-357, or a mutant thereof having all the identifying characteristics thereof; iii) soy protein hydrolysate; iii) chelated ferrous sulfate; and iv) optionally, one or more carriers, excipients, nutrients, or microbial-, biological-, or chemical-crop protection agents, wherein the composition excludes a Bacillus subtilis and wherein the Bacillus licheniformis K-357 is present in an amount suitable to improve plant yield or reduce plant insect damage in the presence of a plant pathogenic insect, or both.

In one embodiment, a plant or a seed of a plant is provided having a coating of a composition comprising: i) a biologically pure culture of a Bacillus licheniformis K-357, or a mutant thereof having all the identifying characteristics thereof; ii) a hydrolysate of soy protein; iii) chelated ferrous sulfate; and iv) optionally one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method of plant or plant seed treatment is provided comprising: applying to a plant or seeds of a plant a coating of a composition comprising: i) a biologically pure culture of a Bacillus licheniformis K-357, or a mutant thereof having all the identifying characteristics thereof; ii) a hydrolysate of soy protein; iii) chelated ferrous sulfate; and iv) optionally one or more carriers, excipients, nutrients, or microbial-, biological-, or chemical-crop protection agents, wherein the composition excludes a Bacillus subtilis and wherein the Bacillus licheniformis K-357 is present in the coating in an amount suitable to improve plant yield or reduce plant insect damage in the presence of a plant pathogenic insect, or both.

In one embodiment, a method is provided of formulating a microbial crop protection agent to improve plant health and/or yield, comprising: adding together a hydrolysate of soy protein, a chelated ferrous sulfate, and a biological culture of one or more microbial crop protection agents, wherein the one or more crop protection agents are present in the formulation in an amount suitable to improve plant yield or reduce plant insect damage in the presence of a plant pathogenic insect, or both.

In one embodiment, a formulation of a microbial crop protection agent is provided to improve plant health and/or yield, comprising: i) a biological culture of one or more microbial crop protection agents; ii) a hydrolysate of soy protein; iii) chelated ferrous sulfate; and iv) optionally, one or more carriers, excipients, nutrients, or non-microbial crop protection agents, wherein the one or more microbial crop protection agents are present in an amount suitable to improve plant yield or reduce plant insect damage in the presence of a plant pathogenic insect, or both.

In the compositions of the present disclosure, the soy protein hydrolysate can comprise about 4% water soluble nitrogen.

In the compositions of the present disclosure, the soy protein hydrolysate can be present in the composition or formulation at about 60% to about 99% by weight and the chelated ferrous sulfate can be present at about 0.05% to about 1.5% by weight.

In the compositions of the present disclosure, the soy protein hydrolysate can be present in the composition or formulation at about 85% to about 95% by weight and the chelated ferrous sulfate can be present at about 0.05% to about 0.3% by weight.

The compositions and formulationsof the present disclosure can be in the form of a liquid and the Bacillus licheniformis K-357 can be present at a concentration ranging from 1.0×10¹² to 1.0×10¹ CFU/ml, 1.0×10¹¹ CFU/ml to 1.0×10² CFU/ml, or from 1.0×10¹⁰ CFU/ml to 1.0×10⁴ CFU/ml.

The compositions and formulations of the present disclosure can be in the form of a dust, a dry wettable powder, a spreadable granule, or a dry wettable granule and the Bacillus licheniformis K-357 can be present in an amount ranging from 1.0×10¹² CFU/g to 1.0×10¹⁰ CFU/g, from 5.0×10¹¹ CFU/g to 1.0×10³ CFU/g, or from 1.25×10¹¹ CFU/g to 1.0×10⁸ CFU/g.

In the methods of the present disclosure, the Bacillus licheniformis K-357 can be applied to or present on the seed in an amount ranging from 1.0×10¹² CFU/g to 1.0×10¹ CFU/g, from 9.0×10⁷ CFU/g to 2.0×10⁴ CFU/g, or from 4.0×10⁸ CFU/g to 1.0×10⁴ CFU/g seed.

Further, in one or more embodiments, suitable insecticides, fungicides, and nematicides of the compositions and methods of the present invention may include: insecticide: A0) agrigata, al-phosphide, ambriseius, apherinus, aphidinus, aphidletes, artemisinin, autographa californica NPV, azocyclotin, Bacillus-subtilis, Bacillus subtilis thuringiensis aizawai (Bacillus-thur.-aizawai), Bacillus thuringiensis kurstakey (Bacillus-thur-kurstaki), Bacillus thuringiensis, beauveria, beauveria-bassiana), Beta-cyfluthrin, biologics, bislutap, broflutrinate, bromophos-e, bromopropyiate, Bt transgenic corn, Bt transgenic soybean, capsaicin, cartap, ceiastrus) Extract, Chloranthraniprole, Chlorbenzuron, Chiorethoxyphos, Chlorfluazuron, Chlorpyrifos-e, Cinidiadin, Cryolite, Cyanophos, Cyantraniprolol, Cyhalothrin, Cihexatin, Cypermethrin, Dakunusa, DCIP, Dichioropropene, Dicophor, Digliphas, Digliphas+Dakunusa, Dimetacarb, Dithioether, Dodecyl Acetate, Emamectin, Encarsia, EPN, Eretmocerus, Ethylene-dibromide, Eucalyptol, Fatty Acid, Salt, Fenazaquin, fenobucarb (BPMC), fenpyroximate, flubrocythrinate, flufensin, formethanate, formotethione, furthiocarb, gamma Cyhaiothrin, garlic juice, granulosis-virus, harmonia, heliothis armigera NPV, inactive bacteria, indol-3-ylbutyric acid, iodomethane, iron, isocarbofos, isofenphos, isofenphos-m, isoprocarb, lsothioate, kaolin, lindane, liuyangmycin, matrine, mephospholane, metaaldehyde, metalidium-anisoprie, methamidophos, metorcarb (MTMC), mineral oil, milex, m-isothiocyanate, monosultap, mirotesium velcaria (Myrothecium verrucaria), nared, honey bee (Neochrysocharis formos), nicotine, nicotinoid, fats and oils, oleic acid, ometoate, orius, Oxymatrine, Pesilomyces, Paraffin oil, Parathion-e, Pasteuria, Petroleum, Pheromone, Phosphate, Photolabdas, Foxime, Phytoseiulus, Pirimiphos-e, Vegetable oil, Plutella xylostella GV, Polyhedrosis virus, polyphenol extract, potassium oleate, profenofos, prosuler, prothiophos, pyracrofos, pyrethrin, pyridafenthione, pyrimidifene, pyriproxyfen, quillay extract, quinomethionate, rapeseed oil, rotenone, Saponin, saponozit, sodium compound, sodium fluorosilicate, starch, steinernema, streptomyces, sulfuramide, sulfur, tebupyrimphos Tefluthrin, temefos, tetradiphone, thiophanox, thiometone, transgenics (eg Cry3Bb1), triazamate, trichoderma, trichogranma, triflumuron, verticillium, beltrin, insecticide isomers (eg kappa-bifenthrin, kappa-tefluthrin), Dichloromesothiaz, brofuranilide, pyradiflumide, various insecticides; A1) carbamates including aidicarb, aranicarb, benfuracarb, carbaryl, carbofuran, carbosulfan, methiocarb, mesomil, oxamyl, pirimicarb, propoxur and thiodicarb; A2) Acephate, azine phos-ethyl, azine phos-methyl, chlorfenvin phos, chlorpyrifos, chlorpyrifos-methyl, de Meton-S-methyl, diazinon, dichlorvos/DDVP, dicrotophos, dimethoate, disulfotone, ethion, fenitrothion, fenthion, isoxathion, malathion, metamidaphos, methidathion, mevinphos, monocrotophos, oxymethoate, oxydemethone-methyl, parathion, parathion-methyl Organophosphates including phentoate, folate, hosalon, phosmet, phosphamidone, pyrimiphos-methyl, quinalphos, terbufos, tetrachlorbinphos, triazophos and trichlorphone; A3) cyclodiene organochloride compounds such as endosulfan; A4) ethiprole, fipronil, Fiprolols including pyrafluprole and pyriprole; A5) acetamiprid, Neonicotinoids including rotianidin, dinotefuran, imidacloprid, nitenpyram, thiacioprid and thiamethoxam; A6) spinosyns such as spinosad and spinetoram; A7) chlorids from mectins including abamectin, emamectin benzoate, ivermectin, lepimectin and milbemectin A8) Juvenile hormone mimetics such as hydroprene, quinoprene, metoprene, phenoxycarb and pyriproxyfen; A9) selective cognate feeding blockers such as pymetrozine, flonicamid and pyrifluquinazone; A10) clofentezine, hexothiazox and etoxazole A11) diafenthiuron, fenbutatin oxide and propargite Mitochondrial ATP synthase inhibitors; oxidative phosphorylation uncouplers such as chlorfenapyr; A12) nicotinic acetylcholine receptor channel blockers such as bensultap, cartap hydrochloride, thiocyclam and thiosuitap sodium; A13) bistriflulone, Type 0 inhibitors of chitin biosynthesis derived from benzoylureas including diflubenzuron, flufenoxuron, hexaflumuron, lufenuron, nobarulone and teflubenzuron; A14) type 1 inhibitors of chitin biosynthesis such as buprofezin; A15) cyromazine A16) ecdysone receptor agonists such as methoxyphenozide, tebufenozide, halofenozide and chromafenozide; A17) octopamine receptors such as amitraz A18) A mitochondrial complex electron transport inhibitor pyridaben, tebufenpyrad, tolfenpyrad, flufenelim, sienopyrafen, cyflumetofene, hydramethylnon, acequinosyl or fluacrylpyrim; A19) voltage-dependent sodium channel blockers such as indoxacarb and metaflumizone; A20) Lipid synthesis inhibitors such as spirodiclofen, spiromesifen and spirotetramat; A21) fulvendiamide, phthalamide compound (R)-3-chloro-N1-{2-methyl-4-[1,2,2,2-tetrafluoro-1-(trifluoromethyl) ethyl] phenyl}-N2-(1-methyl-2-methylsulfonylethyl) phthalamide and (S)-3-chloro-N1-{2-methyl-4 [1,2,2,2-tetrafluoro-1-(trifluoromethyl) ethyl] phenyl}-N2-(1-methyl-2-methylsulfonylethyl) phthalamide, chlorantraniliprole and cyantraniliprole Ryanodine receptor modulators derived from diamides including: A22) compounds with unknown or uncertain mechanisms of action such as azadirachtin, amidoflumet, biphenazate, fluenesulfone, piperonylbutoxide, pyridalyl, sulfoxaflor; or A23) acrinathrin, allethrin, bifenthrin, cyfluthrin, Lambda-cyhalothrin, cypermethrin, alpha-cypermethrin, beta-cypermethrin, zeta-cypermethrin, deltamethrin, esfenvalerate, etofenprox, phen Ropatorin, fenvalerate, flucythrinate, tau-fluvalinate, permethrin, sodium channel modulators derived pyrethroids containing silafluofen and tralomethrin.

Fungicides: B0) benzobindiflupyr, antiperonosporic, amethoctrazine, amisulbrom, copper salts (eg, copper hydroxide, copper acid chloride, copper sulfate, copper persulfate), boscalid, thiflumazide), Fluthianyl, flaxilyl, thiabendazole, benodanyl, mepronyl, isofetamide, fenflam, bixafen, fluxapiroxad, penflufen, sedaxane, succinoxyxine, enoxastrobin, fluphenoxystrobin, pyroxystrobin, pyrame Tostrobin, Triclopyricarb, Phenamine Strobin, Metominostrobin, Pyribencarb, Meptyldinocap, Fentin acetate, Fentin chloride, Fentin hydroxide, Oxytate Cyclin, chlozolinate, chlororiebu, technazen, etridiazole, iodocarb, prothiocarb, Bacillus subtilis synonym, Bacillus amyloliquefaciens (for example, QST 713, FZB24, MBI600, D747 strain), Melaleuca altelni alternifolia extract, Lupinus albus doce extract, BLAD polypeptide, pyrisoxazole, oxpoconazole, etaconazole, fenpyrazamine, naphthifine, terbinafine, validamycin, pyrimorph, varifenalate, Phthalide, probenazole, isotianil, laminarin, Reynoutria sachalinensis extract, phosphorous acid and Salts, teclofthalam, triazoxide, pyriophenone, organic oils, potassium bicarbonate, chlorothalonil, fluoroimide; B1) bittertanol, bromconazole, cyproconazole, difenoconazole, diniconazole, enilconazole, epoxiconazole, Fluquinconazole, fenbuconazole, flusilazole, flutriazole, hexaconazole, imibenconazole, ipconazole, metconazole, microbutanyl, penconazole, propiconazole, prothioconazole, cimeconazole, triadimethone, triadimenol, tebuconazole, tetraco Nazole, triticonazole, prochloraz, pefazoate, imazalyl, triflumizole, cyazofamide, benomyl Carbendazim, thiabendazole, fuberidazole, ethaboxam, etridiazole, hymexazole, azaconazole, diniconazole-M, oxpoconazole, paclobutrazole, uniconazole, 1-(4-chlorophenyl)-2-([1,2,4] triazole-1-yl)-azoles containing cycloheptanol and imazalyl sulfate; B2) azoxystrobin, dimoxystrobin, enestrobrin, fluoxastrobin, cresoxime-methyl, methinostrobin, orisatrobin, picoxy Strobin, pyraclostrobin, trifloxystrobin, enestrobrin, methyl (2-chloro-5-[1-(3-methylbenzyloxyimino) ethyl] benzyl) carbamate, methyl (2-c Ro-5-[1-(6-methylpyridin-2-ylmethoxyimino) ethyl] benzyl) carbamate, methyl 2-(ortho-(2,5-dimethylphenyloxymethylene)-phenyl)-3-methoxyacrylate, 2-(2-(6-(3-Chloro-2-methyl-phenoxy)-5-fluoro-pyrimiclin-4-yloxy)-phenyl)-2-methoxyimino-N-methyl-acetamide and 3-methoxy-2-(2-(N-(4-Methoxy-phenyl)-cyclopropanecarboximidoylsulfanylmethyl)-phenyl)-acrylic acid methyl ester strobilurin; B3) carboxin, benalaxyl, benalaxyl-M, phenhexamide, Flutolanil, furametopil, mepronil, metalaxyl, mefenoxam, Off-race, oxadixyl, oxycarboxyl, penthiopyrad, isopyrazam, tifluzamide, thiazinyl, 3,4-dichloro-N-(2-cyanophenyl) isothiazole-5-carboxamide, dimethomorph, flumorph, flumetober, fluopicolide (picobenzamide), Zoxamide, carpropamide, diclocimet, mandipropamide, N-(2-(4-[3-(4-chlorophenyl) prop-2-ynyloxy]-3-methoxyphenyl) ethyl)-2-methanesulfonyl-amino-3-methylbutylamide, N-(2-(4-[3-(4-chlorophenyl) prop-2-ynyloxy]-3-methoxy-phenyl) ethyl)-2-ethanesulfonylamino-3-methylbutyramide, methyl 3-(4-chloro Enyl)-3-(2-isopropoxycarbonyl-amino-3-methyl-butyrylamino) propionate, N-(4′-bromobiphenyl-2-yl)-4-difluoromethyl-methylthiazole-δ-carboxamide, N-(4′-trifluoromethyl-biphenyl-2-yl)-4-difluoromethyl-2-methylthiazole-5-carboxamide, N-(4′-chloro-3′-fluorobiphenyl-2-yl)-4-Difluoromethyl-2-methyl-thiazole-5-carboxamide, N-(3,4′-dichloro-4-fluorobiphenyl-2-yl)-3-difluoro-methyl-1-methylpyrazole-4-carboxamide, N-(3′,4′-dichloro-5-fluorobiphenyl-2-yl)-3-difluoromethyl-1-methylpyrazo Ru-4-carboxamide, N-(2-cyano-phenyl)-3,4-dichlorolsothlazole-5-carboxamide, 2-amino-4-methyl-thiazole-5-carboxyanilide, 2-chloro-N-(1,1,3-trimethyl-indan-4-yl)-nicotinamide, N-(2-(1,3-dimethyibutyl)-phenyl)-1,3-dimethyl-5-fluoro-1H-pyrazole-4 Carboxamide, N-(4′-chloro-3′,5-difluoro-biphenyl-2-yl)-3-difluoromethyl-1-methyl-1H-pyrazole-4-carboxamide, N-(4′-chloro-3′,5-difluoro-biphenyl-2-y)-3-trifluoromethyl-1-methyl-1H-pyrazole-4-carboxamide, N-(3′,4′-dichloro-5-Fluoro-biphenyl-2-yl)-3-trifluoromethyl-1-methyl-1H-pyrazole-4-carboxamide, N-(3′,5-difluoro-4′-methyl-biphenyl-2-yl)-3-Difluoromethyl-1-methyl-1H-pyrazole-4-carboxamide, N-(3′,5-difluoro-4′-methyl-biphenyl-2-yl)-3-trifluoromethyl-1-methyl-1H-Pyrazole-4-carboxamide, N-(cis-2-blcyclopropyl-2-yl-phenyl)-3-difluoro-methyl-1-methyl-1H-pyrazole-4-carboxamide, N-(trans-2-bicyclopropyl-2-yl-phenyl)-3-difluoro-methyl-1-methyl-1H-pyrazole-4-carboxamide, fluopyram, N-(3-Ethyl-3,5-5-trimethyl-cyclohexyl)-3-formylamino-2-hydroxy-benzamide, oxytetracycline, silthlophane, N-(6-methoxy-pyridin-3-yl) cyclopropanecarboxamide, 2-iodo-N-phenyl-benzamide, N-(2-blcyclo-propyl-2-yl-phenyl)-3-difluoromethyl-1-methylpyrazol-4-ylcarboxamide, N-(3′,4′,5′-Trifluorobiphenyl-2-yl)-1,3-dimethylpyrazo1-4-ylcarboxamide, N-(3′,4′,5′-trifluorobiphenyl-2-yl)-1,3-dimethyl-5 Fluoropyrazol-4-ylcarboxamide, N-(3′,4′,5′-trifluorobiphenyl-2-yl)-5-chloro-1,3-dimethyl-pyrazol-4-ylcarboxamide, N-(3′,4′,5′-trifluorobiphenyl-2-yl)-3-fluoromethyl-1-methylpyrazol-4-ylcarboxamide, N-(3′,4′,5′-trifluorobiphenyl-2-yl)-3-(chlorofluoromethyl)-1-methylpyrazol-4-ylcarboxamide, N-(3′,4′,5′-Trifluorobiphenyl-2-yl)-3-difluoromethyl-1-methylpyrazol-4-ylcarboxamide, N-(3′,4′,5′-trifluorobiphenyl-2-yl)-3-difluoromethyl-5-fluoro-1-methylpyrazol-4-ylcarboxamide, N-(3′,4′,5′-trifluorobiphenyl-2-yl)-5-chlora-3-difluoromethyl-1-methylpyrazol-4-ylcarboxamide, N-(3′,4′,5′-Trifluorobiphenyl-2-yl)-3-(chlorodifluoromethyl)-1-methylpyrazol-4-ylcarboxamide, N-(3′,4′,5′-trifluorobiphenyl-2-yl)-1-methyl-3-influoromethylpyrazal-4-ylcarboxamide, N-(3′,4′,5′-trifluorobiphenyl-2-yl)-5-fluoro-1-methyl-3-trifluoromethyl Pyrazol-4-ylcarboxamide, N-(3′,4′,5′-trifluorobiphenyl-2-yl)-5-chloro-1-methyl-3-trifluoromethylpyrazol-4-ylcarboxyl, N-(2′,4′,5′-trifluorobiphenyl-2-yl)-1,3-dimethylpyrazol-4-ylcarboxamide, N-(2′,4′,5′-trifluorobiphenyl)-2-yl)-1,3-dimethyl-5-fluoropyrazol-4-ylcarboxamide, N-(2′,4′,5′-trifluorobiphenyl-2-yl)-5-chloro-1,3-Dimethylpyrazol-4-ylcarboxamide, N-(2′,4′,5′-trifluorobiphenyl-2-yl)-3-fluoromethyl-1-methylpyrazol-4-ylcarboxamide, N-(2′,4′,5′-trifluorobiphenyl-2-yl)-3-(chlorofluoromethyl)-1-methylpyrazol-4-ylcarboxamide, N-(2′,4′,5′-trifluorobiphenyl-2-yl) 3-difluoromethyl-1-methylpyrazol-4-ylcarboxamide, N-(2′,4′,5″-trifluorobiphenyl-2-yl)-3-difluoromethyl-5-fluoro-1-methylpyrazole-4-ylcarboxamide, N-(2′,4′,5′-trifluorobiphenyl-2-yl)-5-chloro-3-difluoromethyl-1-methylpyrazol-4-ylcarboxamide, N-(2′,4′,5′-trifluorobiphenyl-2-yl)-3-(chlorodifluoromethyl)-1-methylpyrazol-4-ylcarboxamide, N-(2′,4′,5′-trifluorobiphenyl-2-yl)-1-methyl-3-trifluoromethylpyrazol-4-ylcarboxamide, N-(2′,4′,5′-trifluorobiphenyl-2-yl)-5-fur Oro-1-methyl-3-trifluoromethylpyrazol-4-ylcarboxamide, N-(2′,4′,5′-trifluorobiphenyl-2-yl)-5-chloro-1-methyl-3-tri Fluoromethylpyrazol-4-ylcarboxamide, N-(3′,4′-dichloro-3-fluorobiphenyl-2-yl)-1-methyl-3-trifluoromethyl-1H-pyrazole-4-carboxamide, N-(3′,4′-dichloro-3-fluorobiphenyl-2-yl)-1-methyl-3-difluoromethyl-1H-pyrazole-4-carboxamide, N-(3′,4′-difluoro-3-fluoro Biphenyl-2-yl)-1-methyl-3-trifluoromethyl-1H-pyrazole-4-carboxamide, N-(3′,4′-difluoro-3-fluorobiphe Ru-2-yl)-1-methyl-S-difluoromethyl-1H-pyrazole-4-carboxamide, N-(3′-chloro-4′-fluoro-3-fluorobiphenyl-2-yl)-1-methyl-3-Difluoromethyl-1H-pyrazole-4-carboxamide, N-(3′,4′-dichloro-4-fluorobiphenyl-2-yl)-1-methyl-3-trifluoromethyl-1H-pyrazole-4 Carboxamide, N-(3′,4′-difluoro-4-fluorobiphenyl-2-yl)-1-methyl-S-trifluoromethyl-1H-pyrazole-4-carboxamide, N-(3′,4′-Dichloro-4-fluorobiphenyl-2-yl)-1-methyl-3-difluoromethyl-1H-pyrazole-4-carboxamide, N-(3′,4′-difluoro-4 Fluorobiphenyl-2-yl)-1-methyl-3-difluoromethyl-1H-pyrazole-4-carboxamide, N-(3′-chloro-4′-fluoro-4-fluorobiphenyl-2-yl)-1-Methyl-S-difluoromethyl-1H-pyrazole-4-carboxamide, N-(3′,4′-dichloro-5-fluorobiphenyl-2-yl)-1-methyl-3-trifluoromethyl-1H-pyrazole-4-carboxamide, N-(3′,4′-difluoro-5-fluorobiphenyl-2-yl)-1-methyl-3-trifluoromethyl-1H-pyrazole-4-carboxamide, N-(3′,4′-Dichloro-5-fluorobiphenyl-2-yl)-1-methyl-S-difluoromethyl-1H-pyrazole-4-carboxamide, N-(3′,4′-Difluoro-5-fluorobiphenyl-2-yl)-1-methyl-3-difluoromethyl-1H-pyrazole-4-carboxamide, N-(3′,4′-dichloro-5-fluorobiphenyl-2-yl)-1,3-dimethyl-1H-pyrazole-4-carboxamide, N-(3′-chloro-4′-fluoro-5-fluorobiphenyl-2-yl)-1-methyl-3-difluoromethyl-1H-pyrazole-4-carboxamide, N-(4′-fluoro-4-fluorobiphenyl-2-yl)-1-methyl-3-trifluoromethyl-1H-pyrazole-4-carboxamide, N-(4′-fluoro-5-Fluorobibiphenyl-2-yl)-1-methyl-3-trifluoromethyl-1H-pyrazole-4-carboxamide, N-(4′-chloro-S-fur Orobiphenyl-2-yl)-1-methyl-3-trifluoromethyl-1H-pyrazole-4-carboxamide, N-(4′-methyl-5-fluorobiphenyl-2-yl)-1-methyl-3-Trifluoromethyl-1H-pyrazole-4-carboxamide, N-(4′-fluoro-5-fluorobiphenyl-2-yl)-1,3-dimethyl-1H-pyrazole-4-carboxamide, N-(4′-Chloro-5-fluorobiphenyl-2-yl)-1,3-dimethyl-1H-pyrazole-4-carboxamide, N-(4′-methyl-5-fluorobiphenyi-2-yl)-1,3-dimethyl-1H-pyrazole-4-carboxamide, N-(4′-fluoro-6-fluorobiphenyl-2-yl)-1-methyl-3-trifluoromethyl-1H-pi Sol-4-carboxamide, N-(4′-chloro-6-fluorobiphenyl-2-yl)-1-methyl-3-trifluoromethyl-1H-pyrazole-4-carboxamide, N-[2-(1,1,2,3,3,3-hexafluoropropoxy)-phenyl]-3-difluoromethyl-1-methyl-1H-pyrazole-4-carboxamide, N-[4′-(trifluoromethylthio)-biphenyl-2-Yl]-3-difluoromethyl-1-methyl-1H-pyrazole-4-carboxamide and N-[4′-(trifluoromethylthio)-biphenyl-2-yl]-1-methyl-3-trifluoromethyl-carboxamides including 1-methyl-1H-pyrazole-4-carboxamide; B4) fluazinam, pyrifenox, bupyrimeto Prodinil, Phenarimol, Ferimzone, Mepanipyrim, Nuarimol, Pyrimethanyl, Trifolin, Fenpicuronyl, Fludioxonil, Ardimorph, Dodemorph, Fenpropimorph, Tridemorph, Fenpropidin, Iprodione, Procimidone, Vinclozoline, Famoxadone, Fenamiden, Octopronone-Azole, 5-chloro-7-(4-methylpiperidin-1-yl)-6-(2,4,6-trifluorophenyly[1,2,4] triazolo [1,5-a] pyrimidine, Anilazine, dichromedin, pyroxylone, proquinazide, tricyclazole, 2-butoxy-6-iodo-3-propylchromen-4-one, acibenzoral-S-methyl, captahol, captan, dazome, Holpet, phenoxanyl, quinoxyphene, N,N-dimethyl-3-(3-bromo-6-fluoro-2-methylindole-1-sulfonyl)[1,2,4] triazole-1-sulfonamide, -Ethyl-6-octyl-[1,2,4] triazolo [1,5-a] pyrimidine-2,7-diamine, 2,3,5,6-tetrachloro-4-methanesulfonyl-pyridine, 3,4,5-trichloropyridine-2,6-di-carbonitrile, N-(1-(5-bromo-3-chloro-pyridin-2-yl)-ethyl)-2,4-dichloronicotinamide, N-((5-Bromo-3-chloropyridin-2-yl)-methyl)-2,4-dichloro-nicotinamide, diflumetrim, nitrapirine, dodemorph acetate, fluoroimide Heterocyclic compounds including biasticidin-S, quinomethionate, debacarb, diphenzoquat, diphenzoquat-methylsulfate, oxophosphate and piperalin; B5) mancozeb, maneb, metam, metasulfocarb, methylam, felvam, Propinebu, tyram, dineb, ziram, dietofencarb, iprovaricarb, benchavaricarb, propamocarb, propamocarb hydrochloride, 4-fluorophenyl-N-(1-(1-(4-cyanophenyl)-ethanesulfonyl) but 2-yl) carbamate, carbamates including methyl-3-(4-chloro-phenyl)-3-(2-isoproxycarbanylamino-3-methyl-butyrylamino) propanoate; or B6) guanidine, dodine, dodine Free base, iminotadine, guazatine, antibiotics: kasugamycin, streptomycin, polyoxin, validamycin A, nitrophenyl derivatives: binapacryl, dinocup, dinobutone, sulfur-containing heterocyclic compounds: dithianone, isoprothiolane, organometallic compounds: fentin salts, organophosphorus compounds: Edifenphos, iprobenphos, fosetyl, fosetyl-aluminum, phosphorous acid and its salts, pyrazophos, tolcrofos-methyl, organochiorine compounds: dichlorofluanid, fursulfamide, hexachloro-benzene, phthalide, pencyclon, quintozene, thiophanate-methyl, tolylfluani And others: Cyflufenamide, Simoxanyl, Dimiethylylmiol, Ethymol, Flaxil, Metraphenone and Spiroxa, Guazatine-acetate, iminoctadine-triacetate, iminoctadine-tris (albesylate), kasugamycin hydrochloride hydrate, dichlorophen, pentachlorophenol and its salts, N-(4-chloro-2-nitro-phenyl)-N-Ethyl-4-methyl-benzenesulfonamide, dichlorane, nitrotar-isopropyl, technazen, biphenyl, bronopol, diphenylamine, myrdiomycin, oxine copper, prohexadione calcium, N-(cyclopropylmethoxyimino-(6-difluoromethoxy-2,3-difluoro-phenyl)-methyl)-2-phenylacetamide, N′-(4-(4-chloro-3-trifluorolmethyl-phenoxy)-2,5-dimethyl-phenyl)-N-ethyl-N-methylformamidine, N′-(4-(4-fluoro-3-trifluoromethyl-phenoxy)-2,5-dimethyl-phenyl)-N-ethyl-N-methylformamidine, N′-(2-methyl-5-trifluoromethyl-4-(3-trimethylsilanyl-propoxy)-phenyl)-N-ethyl-N-methylformamidine, and N′-(5-difluoromethyl-2-methyl- Other fungicides including 4-(3-trimethylsilanyl-propoxy)-phenyl)-N-ethyl-N-methylformamidine.
Nematicides or bionematicides: benomyl, cloetocarb, aldoxycarb, tilupert, diamidafos, fenamifos, kazusafos, diciofenthion, etoprophos, fensulfothione, phostiazete, heterophos, isamidophos, isazofos, phosphocarbimisiafos, phosphocarbimisiafos Mecalphone, Acetoprole, Benclothiaz, Chloropicrin, Dazomet, Fluenesulfone, 1,3-Dichloropropene (telone), Dimethyl disulfide, Metam sodium, Metam potassium, Metam salt (all produced MITC), Methyl bromide, Biological soil Improvement (eg, mustard seed, mustard seed extract), soil steam fumigation, allyl isothiocyanate (AITC), dimethyl sulfate, Fuaru (aldehyde).
Suitable plant growth regulators of the present invention include: Plant growth regulator: D1) antiauxin such as clofibric acid, 2,3,5-thioclobenzoic acid; D2) 4-CPA, 2,4-D, 2,4-DB, 2,4-DEP, di Chlorprop, fenoprop, IAA, IBA, naphthaleneacetamide, α-naphthaleneacetic acid, 1-naphthol, naphthoxyacetic acid, potassium naphthenate, sodium naphthenate, auxin such as 2,4,5-T; D3) 2iP, benzyladenine Cytokinins such as 4-hydroxyphenethyl alcohol, kinetin and zeatin; D4) dead leaves such as calcium cyanamide, dimethipine, endal, ethephone, melphos, methoxuron, pentachlorophenol, thiazulone and tribuphos; D5) abiglycine and 1-methyl Ethylene such as cyclopropene Inhibitors; D6) Ethylene-releasing agents such as ACC, etaceracyl, etephone, glyoxime; D7) Gametosides such as fenridazone and maleic acid hydrazide; D8) Gibberellins such as gibberellin and gibberellic acid; D9) Abscisic acid, ansimidol, butralin, Carbaryl, chlorophonium, chloroprofam, dikeglac, flumetralin, fluoride amide, fosamine, glyphosdine, isopyrimole, jasmonic acid, maleic hydrazide, mepiquat, piproctanyl, prohydrojasmon, profam, thiaojian, 2,3,5-Growth inhibitors such as triiodobenzoic acid; D10) morphactins such as chlorflurane, chlorfurenol, dichloroflurenol, flulenol; D11) chlormecote, dami Growth inhibitors such as nozide, flurprimidol, mefluidide, paclobutrazole, tetocyciase, uniconazole; D12) growth stimuli such as brassinolide, brassinolide-ethyl, DCPTA, forchiorfenuron, hymexazole, prosuler, triacontanol, etc. Agent: D13) Bacmedesh, benzofluor, buminafos, carvone, choline chloride, siobide, clofenset, cyanamide, cyclanilide, cycloheximide, cyprosulfamide, epocholeon, etizlozate, ethylene, fufenthiourea, fullerane), Heptopargyl, holosulf, inabenfide, caletazan, lead arsenate, metasulfocarb, prohexadione, pida Down, Shintofen, triabenthenol, plant growth regulators that have not been classified such as trinexapac.

In one embodiment, the crop protection agents of the present disclosure can comprise one or a combination of Imidacloprid, Bifenthrin, Metalaxyl, Tebuconazole, Azoxystrobin, Carboxin, Thiram, Fludioxonil, Myclobutanil, Mefenoxam, Sedaxane, Thiamethoxam, Ipconazole or Abamectin.

The compositions, formulations, and methods of the present disclosure can be useful for controlling insect damage from one or more of the following list of plant pathogenic insects including, but not limited to,: plant pathogenic nematodes, Meloidogyne incognita, Heterodera glycines (SCN), Rotylenchulus reniformis, corn rootworm, western corn rootworm (WCR), northern corn rootworm (NCR), southern corn rootworm (SCR), aphids, Green peach aphid (Myzus persicae), Cotton aphid (Aphis gossypii), Diamondback moth (Plutella xylostella), Taro caterpillar (Spodoptera litura), Red flour beetle (Tribolium castaneum), Fall armyworm (Spodoptera frugiperda), and Brown planthopper (Nilaparvata lugens). Cowpea bruchid (Calloso-bruchus maculatus), European cornborer (Ostinia nubilalis), Tobacco hornworm (Manduca sexta), Stem borer (Chilo partellus), Rice brown plant hopper (Nilaparvata lugens), Rice green leaf hopper (Nephotettix cinciteps), Potato leaf hopper (Empoasca fabae), Peach potato aphid (Myzus persicae), Pea aphid (Acyrthosiphon pisum), Gout fly (Chlorop pumilionis), crickets, Locusts, termites, thrips, ants, Mites (Acarina), Lepidopteran (Mamestra brassicae), Wheat bulb fly (Delia coarctata), Cereal aphid (Sitobion avenae), and Colorado potato beetle (Leptinotarsa decemlineata).

In one embodiment, the pathogenic insect comprises one or a combination of plant pathogenic nematodes, Meloidogyne incognita, Heterodera glycines (SCN); Rotylenchulus reniformis, Lesion nematode, corn rootworm, western corn rootworm (WCR), northern corn rootworm (NCR), southern corn rootworm (SCR), aphids, Green peach aphid (Myzus persicae), or Cotton aphid (Aphis gossypii).

### EXAMPLES

### Example 1

### K-357 Inhibits Nematode Hatching In Vitro

An in vitro experiment was performed to evaluate the effect of Bacillus licheniformis K-357 in the control of three different phytonematode species Heterodera glycines, Meloidogyne incognita, and Rotylenchulus reniformis. Nematode eggs were grown in hatching chambers and treated with varying dosage of K-357 spores for 10 days.

The experiment had four treatments:

1. Control: Water

2. K-357@0.0256 1 LJha (corresponding to 5.5×10⁶ CFU in 50 ml)

3. K-357@0.1428 5 L/ha (corresponding to 2.75×10⁷ CFU in 50 ml)

4. K-357@0.3333 10 L/ha (corresponding to 5.5×10⁷ CFU in 50 ml)

Lyophilized K-357 was dispersed in water to provide a concentrate solution (108 CFU/ml), which was diluted in a total of 50 milliliters resulting in the three different concentrations shown above. Each of the 50 ml solutions was applied to the 3 different species of nematode eggs present in petri dishes and left for 10 days. The eggs then underwent microscopic evaluation to obtain hatching counts.

The data are shown in FIGS. 1-5. FIG. 1 is a graph showing the percentage of nematode hatch counts after application of varying dosage of K-357 on species Heterodera glycines, Meloidogyne incognita, and Rotylenchulus reniformis illustrating that K-357 can reduce nematode hatching in a dose-dependent manner, including an 87% reduction of R. reniformis and complete inhibition of M. incognita hatching.

FIGS. 2A-2F are images of nematodes without addition of B. licheniformis K-357 showing: A) nematode eggs, juveniles, and adults; B) unhatched juveniles in eggs; C) a hatching juvenile; D) an adult nematode; E) an adult nematode; and F) mouth of an adult nematode.

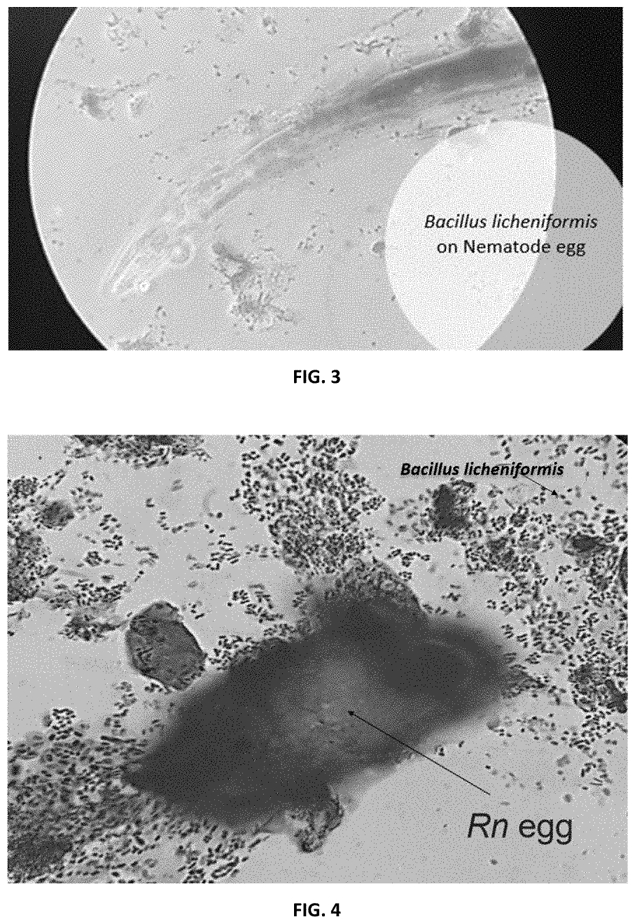

FIG. 3 is an image showing B. licheniformis K-357 colonization of an adult nematode at 1000× magnification.

FIG. 4 is an image at 1000× magnification of B. licheniformis K-357 stained red, surrounding an immobile Reniform nematode egg.

FIG. 5 is an image at 1000× magnification of a Reniform egg surrounded by purple stained B. licheniformis K-357. The juvenile vermiform is visible inside the center of the egg and is immobile under microscopic evaluation.

### Example 2

### K-357-Treated Seed Out-Performs Aldicarb In Cotton Yield

Cotton field trials were conducted at 5 different sites across the United States to evaluate the efficacy of seed treated with B. licheniformis K-357 as nematicide compared to in-furrow application of chemical nematicides Aldicarb (15 lbs/acre) and Fluorpyram (16 oz/acre). The chemical nematicide treatments of Aldicarb and Fluorpyram were applied in-furrow at the time of seed planting. Nematode counts and cotton yield were determined.

The field trial treatment groups were as follows:
1. Fungicide (Azoxystrobin, Fludioxinol, Mefenoxam, Sedaxane)+Insecticide (Thiamethoxam)+Nematicide (Aldicarb 15 lbs/acre)
2. Fungicide (Azoxystrobin, Fludioxinol, Mefenoxam, Sedaxane)+Insecticide (Thiamethoxam, Imidacloprid)+Nematicide (Fluopyram (BAYER) 16 oz/acre)
3. Fungicide (Metalaxyl, Fludioxinol, Myclobutanil)+Insecticide (Imidacloprid)+Nematicide K-357@2.000 Fl. Oz./CWT
   The field trials were performed in the form of a randomized complete block with five or six replicates of each of the treatments (Dependent cooperator setup). The planting population was 2.5 seeds per row ft and the plot size was 4 rows×50 ft.

Method of seed treatment with K-357. The B. licheniformis K-357 treated cotton seed was prepared as follows. Bacillus licheniformis spores were dispersed in a liquid fertilizer solution containing 89.9% by weight soy protein hydrolysate having 4% soluble nitrogen and 0.15% by weight chelated ferrous sulfate to a concentration of 7.0×10⁹ CFU per milliliter (designated “K-357 Liquid”). K-357 Liquid was applied to cotton seed at a rate of two fluid ounces per hundred pounds of cotton seed (designated “2.000 Fl. Oz./CWT”).

Examples of the data from the field trials are shown in FIGS. 6-7.

FIG. 6 is a graph showing the percentage of reniform nematode counts in a field trial in Arkansas 55 days after emergence of cotton seed treated in-furrow with either chemical nematicide Aldicarb or Fluopyram as compared to seed treated with K-357. The results show that B. licheniformis K-357 reduced nematode count to about 70% of the chemical standard, Aldicarb, and out-performed Fluopyram.

FIG. 7 is a graph showing the percentage of cotton yield at harvest for this trial. Surprisingly, the K-357 treated seed resulted in a higher yield of cotton than in furrow treatment with both Fluopyram (˜4.5%) and Aldicarb (˜2.5%).

### Example 3

### K-357-Treated Seed Out-Performs AVICTA ELITE PLUS With VIBRANCE In Cotton Yield

Cotton field trials were conducted at 5 different sites across the United States to evaluate the efficacy of seed treated with B. licheniformis K-357 as nematicide compared to seed treated with AVICTA ELITE PLUS with VIBRANCE (referred to herein as Abamectin) as nematicide in reducing nematode count and increasing yield. In the trial, seed treated with two different rates of B. licheniformis K-357 was compared to seed treated with Abamectin applied at the label rate.

In the field trials, the treated cotton seed groups were as follows:
1. Fungicide (Azoxystrobin, Fludioxinol, Mefenoxam, Sedaxane)+Insecticide (Imidacloprid, Thiamethoxam)+Nematicide (Abamectin, AVICTA ELITE PLUS with VIBRANCE from Syngenta at label rate)
2. Fungicide (Metalaxyl, Fludioxinol, Myclobutanil)+Insecticide (Imidacloprid)+Nematicide (K-357@2.000 Fl. Oz./CWT)
3. Fungicide (Metalaxyl, Fludioxinol, Myclobutanil)+Insecticide (Imidacloprid)+Nematicide (K-357@3.000 Fl. Oz./CWT)
   The field trials were performed in the form of a randomized complete block with five or six replicates of each of the treatments (Dependent cooperator setup). The planting population was 2.5 seeds per row ft and the plot size was 4 rows×50 ft.

Method of seed treatment with K-357. Bacillus licheniformis spores were dispersed in a liquid fertilizer solution containing 89.9% by weight soy protein hydrolysate having 4% soluble nitrogen and 0.15% by weight chelated ferrous sulfate to a concentration of 7.0×109 CFU per milliliter (designated “K-357 Liquid”). K-357 Liquid was applied to cotton seed at a rate of either two or three fluid ounces per hundred pounds of cotton seed (designated “2.000 Fl. Oz./CWT” and “3.000 Fl. Oz./CWT”, respectively).

Examples of the field trial results are shown in FIGS. 8-11.

FIG. 8 is a graph showing the root knot nematode juvenile population at planting, after 60 days, and at harvest in a cotton field trial conducted in Texas comparing seed treated with Abamectin as compared to seed treated with K-357 as nematicide. While the nematode population was lower for Abamectin treated than for K-357 after 60 days, this relationship was reversed by the time of harvest. At harvest the nematode count was reduced to less than 15% for K-357 as opposed to 40% for Abamectin. In fact, the nematode population started out higher in the K-357 group and steadily decreased over the course of the trial. In contrast, the nematode count for the Abamectin group reversed and grew rapidly higher after 60 days. These data demonstrate the more enduring effect on inhibition of K-357 versus Abamectin.

FIG. 9 is a graph showing the percentage of nematode count in a field trial in Mississippi 25 days after planting cotton seed treated with Abamectin or 2- or 3.000 Fl. Oz./CWT of K-357 as nematicide. Surprisingly, the nematode count was ˜8% and ˜12% lower for seed treated with 2.000 Fl. Oz./CWT and 3.000 Fl. Oz./CWT of K-357, respectfully, than for seed treated with Abamectin.

FIG. 10 is a graph showing cotton yield at harvest for the three different seed treatments. The results of treatment with 2.000 Fl. Oz./CWT and 3.000 Fl. Oz./CWT of K-357 on yield were even more dramatic than those for nematode count, resulting in an increase in yield of 54 and 102 lbs/acre, respectively.

FIG. 11 is a graph showing plant vigor calculated on a scale of 1-9, 12 and days after planting cotton seed in the field trial of FIG. 9. The data show that both rates of K-357 improved plant vigor between 12 and 25 days after planting, whereas no improvement in vigor was observed for Abamectin.

### Example 4

### K-357 Kills Nematodes Better than Avicta In Vitro

An in vitro experiment was performed to evaluate the effect of Bacillus licheniformis K-357 on four different phytonematode species Heterodera glycines, Meloidogyne incognita, Rotylenchulus reniformis and Lesion nematode in comparison to the chemical nematicide Avicta. Nematodes were placed in petri dishes and treated with Avicta, water, or K-357 spores for 48 hours as described below.

The experiment had three treatments:

1. Avicta (Syngenta, 0.031%)

2. Control: Water

3. K-357@0.3333 10 L/ha (corresponding to 5.5×10⁷ CFU in 50 ml)

Lyophilized K-357 was dispersed in water to provide a concentrate solution (108 CFU/ml), which was diluted in a total of 50 milliliters. Avicta was similarly diluted into 50 ml and each of the treatments (1)-(3) above was applied to the 4 different species of nematode eggs present in petri dishes and left for 48 hr. Mortality was evaluated by stereoscopic microscopy. The nematodes that showed any mobility or appeared as winding shapes were considered alive and if nematodes did not show any movement and their body shape was straight they were counted as dead.

The data are shown in FIG. 12, which is a graph showing the percentage of nematode mortality for each of the 4 species. Surprisingly, the K-357 treatment was equally or substantially more effective at killing nematodes than Avicta. Specifically, K-357 was equally effective at killing Meloidogyne incognita, Heterodera glycines, and Lesion nematode, and substantially better at killing Rotyienchulus reniformis than Avicta (84.6% versus 35.4%, respectively).

### Example 5

### Corn Field Trials of K-357- and Chemical Insecticide/Fungicide-Treated Corn Seed

Corn field trials were conducted in the United States to evaluate the efficacy of seed treated with B. licheniformis K-357 alone and in combination with chemical insecticides and fungicides for the ability to reduce corn rootworm (CRW) damage and increase yield. The trials were performed at the University of Illinois, Monmouth site, in fields heavily infested with CRW. In the trial, the chemical insecticides and fungicides were applied at the label rate. Corn stand count, root injury mean score, and yield are determined.

In the field trials, the treated corn seed groups were as follows:
1. Fungicide (Azoxystrobin, Metalaxyl, Tebuconazole, Vitavax (Carboxin 37.5%+Thiram 37.5% DS))+Insecticide (Bifenthrin@3.6 FL. Oz./CWT, Imidacloprid@4.8 FL. Oz./CWT)
2. K-357 (2.00 FL. Oz./CWT)+Fungicide (Azoxystrobin, Metalaxyl, Tebuconazole, Vitavax (Carboxin 37.5%+Thiram 37.5% DS))+Insecticide (Bifenthrin@3.6 FL. Oz./CWT, Imidacloprid@4.8 FL. Oz./CWT)
3. K-357 (2.00 FL. Oz./CWT)+Fungicide (Azoxystrobin, Metalaxyl, Tebuconazole, Vitavax (Carboxin 37.5%+Thiram 37.5% DS))
4. K-357 (2.000 Fl. Oz./CWT)
   The field trials were performed in the form of a randomized complete block with four replicates of each of the treatments. The planting population was 4 rows×30 feet (30-inch row spacing) with 5 feet of unplanted alley between plots. Stand count was recorded 37 days after planting and root node-injury score was recorded 64 days after planting.

Method of seed treatment with K-357. Bacillus licheniformis spores were dispersed in liquid fertilizer containing 89.9% by weight soy protein hydrolysate having 4% soluble nitrogen and 0.15% by weight chelated ferrous sulfate to a concentration of 7.0×10⁹ CFU per milliliter (designated “K-357 Liquid”). K-357 Liquid was applied to corn seed at a rate of either two or three fluid ounces per hundred pounds of corn seed (designated “2.000 Fl. Oz./CWT” and “3.000 Fl. Oz./CWT”, respectively).

Stand counts and plant heights were recorded at least once during the early vegetative period. Plots were monitored for early season pests, and if natural infestations occur data are collected across all plots. Corn root systems (5 per plot) were collected, washed, and rated for corn rootworm damage using the 0-3 node-injury scale; the timing of this rating is based on estimates of peak damage determined using degree days and preliminary evaluations of untreated plots (typically mid-late July). Yields are recorded from 2 rows using a plot harvester. Data are subjected to analysis of variance and post-hoc means separation.

The results of stand counts and root node injury analysis 37 days and 64 days after planting, respectively are shown in FIGS. 13 and 14, respectively. The mean stand count for seed treatment group #1 (Insecticide/Fungicide) and seed treatment group #2 (K-357+Insecticide/Fungicide) were both 33.8 (FIG. 13). However, the root node injury graph in FIG. 14 shows that the addition of K-357 to the Insecticide/Fungicide mixture decreased root node injury. Specifically, the root node injury score for seed treatment group #2 (K-357+Insecticide/Fungicide) was 1.12 compared to 1.47 for seed treatment group #1 (Insecticide/Fungicide).

### Example 6

### K-357 Inhibition of Transmission of Potato Virus Y by Green Peach Aphid

The objective of the experiment outlined below is to determine if B. licheniformis K-357 can reduce or prevent potato virus Y (PVY) transmission using a virus transmission assay on Nicotiana benthamiana detached leaves. The green peach aphid (GPA, Myzus persicae) is the most efficient vector of PVY. The hypothesis is that B. licheniformis K-357 is able to debilitate hemptipteran pests such as aphids resulting in complete inhibition and/or reduction of PVY transmission.

Aphids and Virus isolates: A colony of GPA are reared and maintained on a mixture of radish and mustard seedlings in a climate-controlled room (14 h light; 20±3° C.; 70% relative humidity). The strain of PVY used in the study is the recombinant strain, NTN, that was previously collected from infested potato plants in the San Luis Valley, Colo.

Artificial Diet Assays: Feeding chambers are constructed by stretching a piece of parafilm over a petridish (60 mm in diameter). Then, 1.2 ml of artificial diet (Dadd, R. & Mittler, T. Cellular and Molecular Life Sciences 22, 832-833 (1966)) is added and a second layer of stretched of parafilm is placed to form a feeding sachet through which the aphids can feed on the diet. An O-ring is placed on the feeding satchet and aphids are placed on the satchet. The entire set up is covered by the lid of the 60 mm petridish.

Experiment #1: To determine if K-357 acts as an aphidicide, an assay is first performed to determine the mortality of GPA when fed K-357. Briefly, age-synchronized GPA are allowed to feed on artificial diet containing three different concentrations (low, medium and high) of K-357. After 24 and 48 h, the aphids are transferred to detached leaves of N. benthamiana. The growth and mortality rate of the aphids is monitored over a period of 2 weeks. Artificial diet without K-357 serves as control. A concentration of K-357 is used in all future experiments that will cause less than 15% mortality and will be similar to control diet levels.

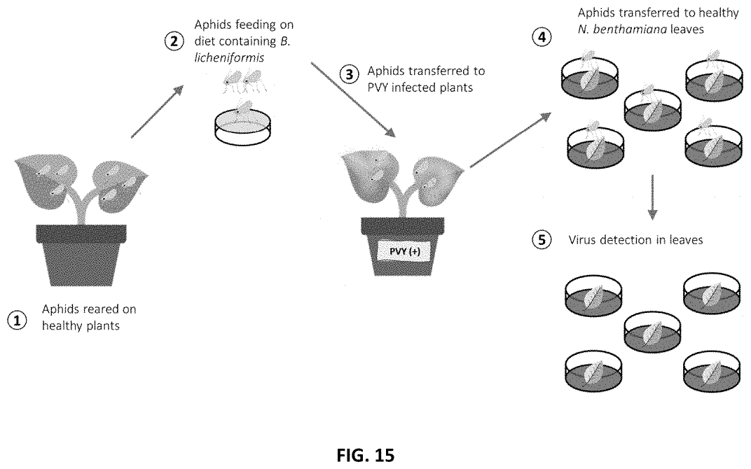

Objective 1: Assay of virus transmission efficiency: To standardize artificial diet experiments, first-instar nymphs (aged 0-24 h) are selected at the onset of each experiment. The experiments for objective 1 are performed in a climate-controlled chamber. A global procedure will be used (see FIG. 15). FIG. 15 illustrates the successive steps of the experimental set-up to investigate the impact of K-357 on the transmission of PVY by the green peach aphid as described below.

Step 1. Age-synchronized aphids are generated by placing 1^{st} instar nymphs on a mix of radish and mustard seedlings.

Step 2. Once the aphids each reach third instar (after 24-48 h), the aphids are transferred onto feeding chambers that contain a concentration of K-357 that causes less than 15% mortality. K-357-free diet serves as control. Aphids are allowed to feed for a period of 24 and 48 h, to ensure that K-357 is taken up by the aphids. For each treatment (4 treatments×2 timepoints of feeding), five age-synchronized GPA are used.

Step 3. After the appropriate time of feeding (24 and 48 h), the GPA are carefully transferred to PVY^{NTN} positive or infected N. benthamiana plants and allowed to feed for 48 h. This period of feeding is the virus acquisition access period or AAP.

Step 4. After a PVY acquisition period of 48 h, individual GPA (one GPA per healthy leaf) is transferred to individual healthy detached N. benthamiana leaves for a 30 min inoculation access feeding period. In the lab and growth room conditions, a single detached leaf of N. benthamiana placed in a petri dish with wet filter paper will survive without any detrimental effects for up to a month. The individual GPA is removed, collected and stored for further analysis (if required).

Step 5. The development of symptoms on the detached N. benthamiana leaves is monitored daily over a period of three weeks. At the onset of symptom development, a small portion of the leaf is collected and tested for the presence of PVY using an IMMUNOSTRIP for PVY (AGDIA, Inc., Indiana).

All the tests are repeated five times with 10 replicates containing one aphid (total: 400 individual GPA and detached N. benthamiana leaves). The influence of K-357 on virus transmission (number of virus infected plants by batch of plants) is determined with ANOVA analysis.

Accordingly, while the methods and systems have been described in reference to specific embodiments, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein.

Various combinations and sub-combinations of the compositions, methods, and features described herein are contemplated and will be apparent to a skilled person having knowledge of this disclosure. Any of the various features and elements as disclosed herein may be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

What is claimed is:

1. A method for controlling insect damage to a plant, the method comprising:
planting a plant or a seed of a plant in a suitable growth medium, the plant or the seed having a coating or partial coating of a composition comprising:
a biologically pure culture of a Bacillus licheniformis K-357, or a mutant thereof having all the identifying characteristics thereof; and
optionally, one or more carriers, excipients, nutrients, or microbial-, biological-, or chemical-crop protection agents,
wherein the composition excludes a Bacillus subtilis and wherein the Bacillus licheniformis K-357 is present in an amount suitable to improve plant yield or reduce plant insect damage in the presence of a plant pathogenic insect, or both.

2. The method of claim 1, wherein the Bacillus licheniformis K-357 is present on the seed in an amount ranging from 1.0×10¹² CFU/g to 1.0×10¹ CFU/g, from 9.0×10⁷ CFU/g to 2.0×10⁴ CFU/g, or from
4.0×10⁸ CFU/g to 1.0×10⁴ CFU/g seed.

3. The method of claim 1, wherein the crop protection agents comprise one or a combination of Imidacloprid, Bifenthrin, Metalaxyl, Tebuconazole, Azoxystrobin, Carboxin, Thiram, Fludioxonil, Myclobutanil, Mefenoxam, Sedaxane, Thiamethoxam, Ipconazole or Abamectin.

4. The method of claim 1, wherein the insect comprises plant pathogenic nematodes, Meloidogyne incognita, Heterodera glycines (SCN), Rotyienchuius renifonnis, Lesion nematode, corn rootworm, western corn rootworm (WCR), northern corn rootworm (NCR), southern corn rootworm (SCR), aphids, Green peach aphid (Myzus persicae), or Cotton aphid (Aphis gossypii).

5. A method for controlling insect damage to a plant, the method comprising:
delivering to seed of a plant, foliage of a plant, roots of a plant, or soil or growth medium surrounding a plant, a composition comprising:
a biologically pure culture of a Bacillus licheniformis K-357, or a mutant thereof having all the identifying characteristics thereof; and
optionally, one or more carriers, excipients, nutrients, or microbial-, biological-, or chemical-crop protection agents,
wherein the composition excludes a Bacillus subtilis and wherein the Bacillus licheniformis K-357 is present in an amount suitable to improve plant yield or reduce plant insect damage in the presence of a plant pathogenic insect, or both.

6. The method of claim 5, wherein the composition is in the form of a liquid and the Bacillus licheniformis K-357 is present at a concentration ranging from 1.0×10¹² to 1.0×10¹ CFU/ml, 1.0×10¹¹ CFU/ml to 1.0×10² CFU/ml, or from 1.0×10¹⁰ CFU/ml to 1.0×10⁴ CFU/ml.

7. The method of claim 5, wherein the composition is in the form of a dust, a dry wettable powder, a spreadable granule, or a dry wettable granule and the Bacillus licheniformis K-357 is present in an amount ranging from 1.0×10¹² CFU/g to 1.0×10¹⁰ CFU/g, from 5.0×10¹¹ CFU/g to 1.0×10³ CFU/g, or from 1.25×10¹¹ CFU/g to 1.0×10⁸ CFU/g.

8. The method of claim 5, wherein the plant comprises corn vegetables, monocots, dicots, cotton, rice, soybean, tomato, cereals, root/tuber and brassica vegetables, cucurbit vegetables, bulb vegetables, citrus, fruiting vegetables, herbs/spices, leafy vegetables, legumes/vegetables (succulent and dried beans and peas), oil seed crops, sunflower, pome fruit, stone fruit, strawberry, sugarcane, sugarbeet, tree nuts, kiwi, banana, grass, ornamental plants, or hardwood cuttings.

9. The method of claim 5, wherein the insect comprises plant pathogenic nematodes, Meloidogyne incognita, Heterodera glycines (SCN), Rotylenchultis reniformis, Lesion nematode, corn rootworm, western corn rootworm (WCR), northern corn rootworm (NCR), southern corn rootworm (SCR), aphids, Green peach aphid (Myzus persicae), or Cotton aphid (Aphis gossypii).

10. A method of plant or plant seed treatment comprising applying to a plant or seeds of a plant a coating of a composition comprising:
a biologically pure culture of a Bacillus licheniformis K-357, or a mutant thereof having all the identifying characteristics thereof; and
optionally, one or more carriers, excipients, nutrients, or microbial-, biological-, or chemical-crop protection agents,
wherein the composition excludes a Bacillus subtilis and wherein the Bacillus licheniformis K-357 is present in the coating in an amount suitable to improve plant yield or reduce plant insect damage in the presence of a plant pathogenic insect, or both.

11. The method of claim 10, wherein the Bacillus licheniformis K-357 is applied to the seed in an amount ranging from 1.0×10¹² CFU/g to 1.0×10¹ CFU/g, from 9.0×10⁷ CFU/g to 2.0×10⁴ CFU/g, or from 4.0×10⁸ CFU/g to 1.0×10⁴ CFU/g seed.

12. The method of claim 10, wherein the coating is applied to the plant foliage, the plant root tips, or the plant transplant roots.

13. A plant or a seed of a plant having a coating of a composition comprising:
a biologically pure culture of a Bacillus licheniformis K-357 or a mutant thereof having all the identifying characteristics thereof; and
optionally, one or more carriers, excipients, nutrients, or microbial-, biological- or chemical-crop protection agents,
wherein the composition excludes a Bacillus subtilis and wherein the Bacillus licheniformis K-357 is present in the coating in an amount suitable to improve plant yield or reduce plant insect damage in the presence of a plant pathogenic insect, or both.

14. The plant or seed of claim 13, wherein the Bacillus licheniformis K-357 is present on the plant or seed in an amount ranging from 1.0×10¹² CFU/g to 1.0×10¹ CFU/g, from 9.0×10⁷ CFU/g to 2.0×10⁴ CFU/g, or from 4.0×10⁸ CFU/g to 1.0×10⁴ CFU/g seed.

15. The plant or seed of claim 13, wherein the crop protection agent comprises one or a combination of Imidacloprid, Bifenthrin, Metalaxyl, Tebuconazole, Azoxystrobin, Carboxin, Thiram, Fludioxonil, Myclobutanil, Mefenoxam, Sedaxane, Thiamethoxam, Ipconazole or Abamectin.

16. The plant or seed of claim 13, wherein the plant foliage, the plant root tips, or the plant transplant roots have the coating.

17. The plant or seed of claim 13, wherein the plant comprises corn vegetables, monocots, dicots, cotton, rice, soybean, tomato, cereals, root/tuber and brassica vegetables, cucurbit vegetables, bulb vegetables, citrus, fruiting vegetables, herbs/spices, leafy vegetables, legumes/vegetables (succulent and dried beans and peas), oil seed crops, sunflower, pome fruit, stone fruit, strawberry, sugarcane, sugarbeet, tree nuts, kiwi, banana, grass, ornamental plants, or hardwood cuttings.

18. The plant or seed of claim 13, wherein the insect comprises plant pathogenic nematodes, Meloidogyne incognita, Heterodera glycines (SCN), Rotylenchulus reniformis, Lesion nematode, corn rootworm, western corn rootworm (WCR), northern corn rootworm (NCR), southern corn rootworm (SCR), aphids, Green peach aphid (Myzus persicae), or Cotton aphid (Aphis gossypii).

19. The plant or seed of claim 13, wherein the composition comprises a hydrolysate of soy protein and chelated ferrous sulfate.

20. A composition for improving plant health and/or yield comprising:
i. a biologically pure culture of a Bacillus licheniformis K-357, or a mutant thereof having all the identifying characteristics thereof;
ii. soy protein hydrolysate;
iii. chelated ferrous sulfate; and
iv. optionally, one or more carriers, excipients, nutrients, or microbial-, biological-, or chemical-crop protection agents,
wherein the composition excludes a Bacillus subtilis and wherein the Bacillus licheniformis K-357 is present in an amount suitable to improve plant yield or reduce plant insect damage in the presence of a plant pathogenic insect, or both.
